(19) United States
(12) Patent Application Publication
(10) Pub. No.: US 2025/0381266 A1
(43) Pub. Date: Dec. 18, 2025

(54) IMMUNOTHERAPEUTIC COMPOSITIONS FOR TREATMENT OF GLIOBLASTOMA MULTIFORME

(71) Applicant: Variation Biotechnologies Inc., Ottawa (CA)

(72) Inventor: David Evander Anderson, Newton, MA (US)

(21) Appl. No.: 19/002,186

(22) Filed: Dec. 26, 2024

Related U.S. Application Data
(63) Continuation of application No. 16/888,398, filed on May 29, 2020, now abandoned.
(60) Provisional application No. 62/855,120, filed on May 31, 2019.

(51) Int. Cl. A61K 39/245; A61K 39/00; A61P 35/00; C07K 14/005; C12N 7/00

(52) U.S. Cl. CPC A61K 39/245; A61P 35/00; C07K 14/005; C12N 7/00; A61K 2039/5256; A61K 2039/5258; A61K 2039/545; A61K 2039/55522; A61K 2039/575; A61K 2039/585; A61K 2039/70; A61K 2039/80; C07K 2319/00; C12N 2710/16122; C12N 2710/16134; C12N 2710/16171; C12N 2740/13022; C12N 2740/13034; C12N 2740/13071

(57) ABSTRACT

The present disclosure provides compositions and methods useful for treating Glioblastoma Multiforme (GBM), e.g., compositions comprising virus-like particles (VLPs) comprising Moloney Murine leukemia virus (MMLV) core proteins and the human cytomegalovirus epitopes, gB and pp65, formulated with GM-CSF, which, at dose of at least 10 μg gB/pp65Gag, reverse dysregulation of anti-HCMV immunity in GBM patients.

### CROSS REFERENCE TO RELATED APPLICATIONS

The present application is a continuation application of U.S. application Ser. No. 16/888,398 filed May 29, 2020, which claims the benefit of U.S. Prov. Appln. No. 62/855,120, filed May 31, 2019, the entire contents of which are incorporated by reference herein in their entirety.

### SEQUENCE LISTING

The instant application contains a Sequence Listing which has been submitted electronically in XML format and is hereby incorporated by reference in its entirety. Said XML copy, created on Aug. 22, 2025, is named “2007801-0163_SL.xml” and is 33,366 bytes in size.

### FIELD OF THE INVENTION

This invention is in the field of immune-oncology, in particular virus like particle vaccines for use in the treatment of Glioblastoma Multiforme.

### BACKGROUND

Glioblastoma Multiforme (GBM) is the most common and aggressive primary form of brain tumour with median survival time being only three months without treatment. GBM affects 2 to 3 adults per 100,000 each year in the United States and Europe. In the United States alone each year, GBM is diagnosed in more than 20,000 people and is responsible for about 15,000 deaths.

### SUMMARY

The present disclosure provides compositions and methods useful for treatment of GBM. More particularly, the present disclosure provides compositions comprising virus like particles (VLPs) expressing antigens from HCMV and methods for their use. The compositions of the invention comprise VLPs expressing the HCMV antigens gB and pp65.

In a preferred embodiment of the invention, the compositions of the invention comprise pp65-gB VLPs formulated with granulocyte macrophage colony stimulating factor (“GM-CSF”) as an adjuvant.

In a preferred embodiment of the invention, the compositions of the invention comprise pp65-gB VLPs formulated with GM-CSF as an adjuvant in a dose of at least about 0.4 μg pp65 and about 200 μg GM-CSF. In another embodiment of the invention, the compositions of the invention comprise pp65-gB VLPs formulated with GM-CSF as an adjuvant in a dose of at least about 10 μg pp65 and about 200 μg GM-CSF

The present disclosure also provides methods of treatment of patients suffering from GBM, the method comprising administration of the compositions of the invention by intradermal injection. In a preferred embodiment, the injections are provided as two half dose injections at separate sites. In a particularly preferred embodiment, the injections are provided as two half dose injections at separate sites, on a monthly basis. In a further embodiment, the present disclosure provides methods of treatment of patients suffering from GBM wherein the patients demonstrate dysregulation of immunity to HCMV, the method comprising administration of the composition of the invention at doses of at least about 10 μg pp65 and about 200 μg GM-CSF.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### LISTING OF SEQUENCES

The following is a list of sequences referred to herein:

SEQ ID NO: 1 is an MMLV-Gag Amino Acid Sequence
MGQTVTTPLSLTLGHWKDVERIAHNQSVDVKKRRWVTFCSAEWPTFNVGWPRDGTFN
RDLITQVKIKVFSPGPHGHPDQVPYIVTWEALAFDPPPWVKPFVHPKPPPPLPPSAPSLPL
EPPRSTPPRSSLYPALTPSLGAKPKPQVLSDSGGPLIDLLTEDPPPYRDPRPPPSDRDGNGG
EATPAGEAPDPSPMASRLRGRREPPVADSTTSQAFPLRAGGNGQLQYWPFSSSDLYNW
KNNNPSFSEDPGKLTALIESVLITHQPTWDDCQQLLGTLLTGEEKQRVLLEARKAVRGD
DGRPTQLPNEVDAAFPLERPDWDYTTQAGRNHLVHYRQLLLAGLQNAGRSPTNLAKV
KGITQGPNESPSAFLERLKEAYRRYTPYDPEDPGQETNVSMSFIWQSAPDIGRKLERLED
LKNKTLGDLVREAEKIFNKRETPEEREERIRRETEEKEERRRTEDEQKEKERDRRRHREM
SKLLATVVSGQKQDRQGGERRRSQLDRDQCAYCKEKGHWAKDCPKKPRGPRGPRPQT
SLLTLDD
SEQ ID NO: 2 is MMLV-Gag Nucleotide Sequence
ATGGGCCAGACTGTTACCACTCCCTTAAGTTTGACCTTAGGTCACTGGAAAGATGTC
GAGCGGATCGCTCACAACCAGTCGGTAGATGTCAAGAAGAGACGTTGGGTTACCTT
CTGCTCTGCAGAATGGCCAACCTTTAACGTCGGATGGCCGCGAGACGGCACCTTTAA
CCGAGACCTCATCACCCAGGTTAAGATCAAGGTCTTTTCACCTGGCCCGCATGGACA
CCCAGACCAGGTCCCCTACATCGTGACCTGGGAAGCCTTGGCTTTTGACCCCCCTCC
CTGGGTCAAGCCCTTTGTACACCCTAAGCCTCCGCCTCCTCTTCCTCCATCCGCCCCG
TCTCTCCCCCTTGAACCTCCTCGTTCGACCCCGCCTCGATCCTCCCTTTATCCAGCCC
TCACTCCTTCTCTAGGCGCCAAACCTAAACCTCAAGTTCTTTCTGACAGTGGGGGGC
CGCTCATCGACCTACTTACAGAAGACCCCCCGCCTTATAGGGACCCAAGACCACCCC
CTTCCGACAGGGACGGAAATGGTGGAGAAGCGACCCCTGCGGGAGAGGCACCGGA
CCCCTCCCCAATGGCATCTCGCCTACGTGGGAGACGGGAGCCCCCTGTGGCCGACTC
CACTACCTCGCAGGCATTCCCCCTCCGCGCAGGAGGAAACGGACAGCTTCAATACT
GGCCGTTCTCCTCTTCTGACCTTTACAACTGGAAAAATAATAACCCTTCTTTTTCTGA
AGATCCAGGTAAACTGACAGCTCTGATCGAGTCTGTTCTCATCACCCATCAGCCCAC
CTGGGACGACTGTCAGCAGCTGTTGGGGACTCTGCTGACCGGAGAAGAAAAACAAC
GGGTGCTCTTAGAGGCTAGAAAGGCGGTGCGGGGCGATGATGGGCGCCCCACTCAA
CTGCCCAATGAAGTCGATGCCGCTTTTCCCCTCGAGCGCCCAGACTGGGATTACACC
ACCCAGGCAGGTAGGAACCACCTAGTCCACTATCGCCAGTTGCTCCTAGCGGGTCTC
CAAAACGCGGGCAGAAGCCCCACCAATTTGGCCAAGGTAAAAGGAATAACACAAG
GGCCCAATGAGTCTCCCTCGGCCTTCCTAGAGAGACTTAAGGAAGCCTATCGCAGGT
ACACTCCTTATGACCCTGAGGACCCAGGGCAAGAAACTAATGTGTCTATGTCTTTCA
TTTGGCAGTCTGCCCCAGACATTGGGAGAAAGTTAGAGAGGTTAGAAGATTTAAAA
AACAAGACGCTTGGAGATTTGGTTAGAGAGGCAGAAAAGATCTTTAATAAACGAGA
AACCCCGGAAGAAAGAGAGGAACGTATCAGGAGAGAAACAGAGGAAAAAGAAGA
ACGCCGTAGGACAGAGGATGAGCAGAAAGAGAAAGAAAGAGATCGTAGGAGACAT
AGAGAGATGAGCAAGCTATTGGCCACTGTCGTTAGTGGACAGAAACAGGATAGACA
GGGAGGAGAACGAAGGAGGTCCCAACTCGATCGCGACCAGTGTGCCTACTGCAAAG
AAAAGGGGCACTGGGCTAAAGATTGTCCCAAGAAACCACGAGGACCTCGGGGACC
AAGACCCCAGACCTCCCTCCTGACCCTAGATGAC
SEQ ID NO: 3 is a Codon Optimized MMLV-Gag Nucleotide
Sequence
(SEQ ID NO: 3)
ATGGGACAGACCGTCACAACACCCCTGAGCCTGACCCTGGGACATTGGAAAGACGT
GGAGAGGATCGCACATAACCAGAGCGTGGACGTGAAGAAACGGAGATGGGTCACA
TTCTGCAGTGCTGAGTGGCCAACTTTTAATGTGGGATGGCCCCGAGACGGCACTTTC
AACAGGGATCTGATCACCCAGGTGAAGATCAAGGTCTTTAGCCCAGGACCTCACGG
ACATCCAGACCAGGTGCCTTATATCGTCACCTGGGAGGCACTGGCCTTCGATCCCCC
TCCATGGGTGAAGCCATTTGTCCACCCAAAACCACCTCCACCACTGCCTCCAAGTGC
CCCTTCACTGCCACTGGAACCACCCCGGAGCACACCACCCCGCAGCTCCCTGTATCC
TGCTCTGACTCCATCTCTGGGCGCAAAGCCAAAACCACAGGTGCTGAGCGACTCCG
GAGGACCACTGATTGACCTGCTGACAGAGGACCCCCCACCATACCGAGATCCTCGG
CCTCCACCAAGCGACCGCGATGGAAATGGAGGAGAGGCTACTCCTGCCGGCGAAGC
CCCTGACCCATCTCCAATGGCTAGTAGGCTGCGCGGCAGGCGCGAGCCTCCAGTGG
CAGATAGCACCACATCCCAGGCCTTCCCTCTGAGGGCTGGGGGAAATGGGCAGCTC
CAGTATTGGCCATTTTCTAGTTCAGACCTGTACAACTGGAAGAACAATAACCCCTCT
TTCAGTGAGGACCCCGGCAAACTGACCGCCCTGATCGAATCCGTGCTGATTACCCAT
CAGCCCACATGGGACGATTGTCAGCAGCTCCTGGGCACCCTGCTGACCGGAGAGGA
AAAGCAGCGCGTGCTGCTGGAGGCTCGCAAAGCAGTCCGAGGGGACGATGGACGG
CCCACACAGCTCCCTAATGAGGTGGACGCCGCTTTTCCACTGGAAAGACCCGACTGG
GATTATACTACCCAGGCAGGGAGAAACCACCTGGTCCATTACAGGCAGCTCCTGCT
GGCAGGCCTGCAGAATGCCGGGAGATCCCCCACCAACCTGGCCAAGGTGAAAGGCA
TCACACAGGGGCCTAATGAGTCACCAAGCGCCTTTCTGGAGAGGCTGAAGGAAGCT
TACCGACGGTATACCCCATACGACCCTGAGGACCCCGGACAGGAAACAAACGTCTC
CATGTCTTTCATCTGGCAGTCTGCCCCAGACATTGGGCGGAAGCTGGAGAGACTGGA
AGACCTGAAGAACAAGACCCTGGGCGACCTGGTGCGGGAGGCTGAAAAGATCTTCA
ACAAACGGGAGACCCCCGAGGAAAGAGAGGAAAGGATTAGAAGGGAAACTGAGGA
AAAGGAGGAACGCCGACGGACCGAGGACGAACAGAAGGAGAAAGAACGAGATCG
GCGGCGGCACCGGGAGATGTCAAAGCTGCTGGCCACCGTGGTCAGCGGACAGAAAC
AGGACAGACAGGGAGGAGAGCGACGGAGAAGCCAGCTCGACAGGGATCAGTGCGC
ATACTGTAAGGAAAAAGGCCATTGGGCCAAGGATTGCCCCAAAAAGCCAAGAGGAC
CAAGAGGACCAAGACCACAGACATCACTGCTGACCCTGGACGAC
SEQ ID NO: 4 is a MMLV Gag - CMV pp65 Amino Acid Sequence
(SEQ ID NO: 4)
MGQTVTTPLSLTLGHWKDVERIAHNQSVDVKKRRWVTFCSAEWPTFNVGWPRDG
TFNRDLITQVKIKVFSPGPHGHPDQVPYIVTWEALAFDPPPWVKPFVHPKPPPPLPP
SAPSLPLEPPRSTPPRSSLYPALTPSLGAKPKPQVLSDSGGPLIDLLTEDPPPYRDPRP
PPSDRDGNGGEATPAGEAPDPSPMASRLRGRREPPVADSTTSQAFPLRAGGNGQLQ
YWPFSSSDLYNWKNNNPSFSEDPGKLTALIESVLITHQPTWDDCQQLLGTLLTGEE
KQRVLLEARKAVRGDDGRPTQLPNEVDAAFPLERPDWDYTTQAGRNHLVHYRQL
LLAGLQNAGRSPTNLAKVKGITQGPNESPSAFLERLKEAYRRYTPYDPEDPGQETN
VSMSFIWQSAPDIGRKLERLEDLKNKTLGDLVREAEKIFNKRETPEEREERIRRETE
EKEERRRTEDEQKEKERDRRRHREMSKLLATVVSGQKQDRQGGERRRSQLDRDQ
CAYCKEKGHWAKDCPKKPRGPRGPRPQTSLLTLDDCESRGRRCPEMISVLGPISGHV
LKAVFSRGDTPVLPHETRLLQTGIHVRVSQPSLILVSQYTPDSTPCHRGDNQLQVQHTYF
TGSEVENVSVNVHNPTGRSICPSQEPMSIYVYALPLKMLNIPSINVHHYPSAAERKHRHL
PVADAVIHASGKQMWQARLTVSGLAWTRQQNQWKEPDVYYTSAFVFPTKDVALRHV
VCAHELVCSMENTRATKMQVIGDQYVKVYLESFCEDVPSGKLEMHVTLGSDVEEDLT
MTRNPQPFMRPHERNGFTVLCPKNMIIKPGKISHIMLDVAFTSHEHFGLLCPKSIPGLSIS
GNLLMNGQQIFLEVQAIRETVELRQYDPVAALFFEDIDLLLQRGPQYSEHPTFTSQYRIQ
GKLEYRHTWDRHDEGAAQGDDDVWTSGSDSDEELVTTERKTPRVTGGGAMAGASTSA
GRKRKSASSATACTAGVMTRGRLKAESTVAPEEDTDEDSDNEIHNPAVETWPPWQAGI
LARNLVPMVATVQGQNLKYQEFFWDANDIYRIFAELEGVWQPAAQPKRRRHRQDALP
GPCIASTPKKHRG* (MMLV Gag amino acid sequence bolded)
SEQ ID NO: 5 is a MMLV Gag - CMV pp65 Nucleotide Sequence
(SEQ ID NO: 5)
ATGGGCCAGACTGTTACCACTCCCTTAAGTTTGACCTTAGGTCACTGGAAAGAT
GTCGAGCGGATCGCTCACAACCAGTCGGTAGATGTCAAGAAGAGACGTTGGGT
TACCTTCTGCTCTGCAGAATGGCCAACCTTTAACGTCGGATGGCCGCGAGACG
GCACCTTTAACCGAGACCTCATCACCCAGGTTAAGATCAAGGTCTTTTCACCTG
GCCCGCATGGACACCCAGACCAGGTCCCCTACATCGTGACCTGGGAAGCCTTG
GCTTTTGACCCCCCTCCCTGGGTCAAGCCCTTTGTACACCCTAAGCCTCCGCCT
CCTCTTCCTCCATCCGCCCCGTCTCTCCCCCTTGAACCTCCTCGTTCGACCCCG
CCTCGATCCTCCCTTTATCCAGCCCTCACTCCTTCTCTAGGCGCCAAACCTAAA
CCTCAAGTTCTTTCTGACAGTGGGGGGCCGCTCATCGACCTACTTACAGAAGA
CCCCCCGCCTTATAGGGACCCAAGACCACCCCCTTCCGACAGGGACGGAAATG
GTGGAGAAGCGACCCCTGCGGGAGAGGCACCGGACCCCTCCCCAATGGCATCT
CGCCTACGTGGGAGACGGGAGCCCCCTGTGGCCGACTCCACTACCTCGCAGGC
ATTCCCCCTCCGCGCAGGAGGAAACGGACAGCTTCAATACTGGCCGTTCTCCT
CTTCTGACCTTTACAACTGGAAAAATAATAACCCTTCTTTTTCTGAAGATCCAG
GTAAACTGACAGCTCTGATCGAGTCTGTTCTCATCACCCATCAGCCCACCTGGG
ACGACTGTCAGCAGCTGTTGGGGACTCTGCTGACCGGAGAAGAAAAACAACGG
GTGCTCTTAGAGGCTAGAAAGGCGGTGCGGGGCGATGATGGGCGCCCCACTCA
ACTGCCCAATGAAGTCGATGCCGCTTTTCCCCTCGAGCGCCCAGACTGGGATT
ACACCACCCAGGCAGGTAGGAACCACCTAGTCCACTATCGCCAGTTGCTCCTA
GCGGGTCTCCAAAACGCGGGCAGAAGCCCCACCAATTTGGCCAAGGTAAAAGG
AATAACACAAGGGCCCAATGAGTCTCCCTCGGCCTTCCTAGAGAGACTTAAGG
AAGCCTATCGCAGGTACACTCCTTATGACCCTGAGGACCCAGGGCAAGAAACT
AATGTGTCTATGTCTTTCATTTGGCAGTCTGCCCCAGACATTGGGAGAAAGTTA
GAGAGGTTAGAAGATTTAAAAAACAAGACGCTTGGAGATTTGGTTAGAGAGGC
AGAAAAGATCTTTAATAAACGAGAAACCCCGGAAGAAAGAGAGGAACGTATCA
GGAGAGAAACAGAGGAAAAAGAAGAACGCCGTAGGACAGAGGATGAGCAGAA
AGAGAAAGAAAGAGATCGTAGGAGACATAGAGAGATGAGCAAGCTATTGGCCA
CTGTCGTTAGTGGACAGAAACAGGATAGACAGGGAGGAGAACGAAGGAGGTC
CCAACTCGATCGCGACCAGTGTGCCTACTGCAAAGAAAAGGGGCACTGGGCTA
AAGATTGTCCCAAGAAACCACGAGGACCTCGGGGACCAAGACCCCAGACCTCC
CTCCTGACCCTAGATGACTGTGAGTCGCGCGGTCGCCGTTGTCCCGAAATGATATC
CGTACTGGGTCCCATTTCGGGGCACGTGCTGAAAGCCGTGTTTAGTCGCGGCGACAC
GCCGGTGCTGCCGCACGAGACGCGACTCCTGCAGACGGGTATCCACGTGCGCGTGA
GCCAGCCCTCGCTGATCCTGGTGTCGCAGTACACGCCCGACTCGACGCCATGCCACC
GCGGCGACAATCAGCTGCAGGTGCAGCACACGTACTTTACGGGCAGCGAGGTGGAG
AACGTGTCGGTCAACGTGCACAACCCCACGGGCCGGAGCATCTGCCCCAGCCAAGA
GCCCATGTCGATCTATGTGTACGCGCTGCCGCTCAAGATGCTGAACATCCCCAGCAT
CAACGTGCACCACTACCCGTCGGCGGCCGAGCGCAAACACCGACACCTGCCCGTAG
CTGACGCTGTGATTCACGCGTCGGGCAAGCAGATGTGGCAGGCGCGTCTCACGGTCT
CGGGACTGGCCTGGACGCGTCAGCAGAACCAGTGGAAAGAGCCCGACGTCTACTAC
ACGTCAGCGTTCGTGTTTCCCACCAAGGACGTGGCACTGCGGCACGTGGTGTGCGCG
CACGAGCTGGTTTGCTCCATGGAGAACACGCGCGCAACCAAGATGCAGGTGATAGG
TGACCAGTACGTCAAGGTGTACCTGGAGTCCTTCTGCGAGGACGTGCCCTCCGGCAA
GCTCTTTATGCACGTCACGCTGGGCTCTGACGTGGAAGAGGACCTGACGATGACCCG
CAACCCGCAACCCTTCATGCGCCCCCACGAGCGCAACGGCTTTACGGTGTTGTGTCC
CAAAAATATGATAATCAAACCGGGCAAGATCTCGCACATCATGCTGGATGTGGCTTT
TACCTCACACGAGCATTTTGGGCTGCTGTGTCCCAAGAGCATCCCGGGCCTGAGCAT
CTCAGGTAACCTATTGATGAACGGGCAGCAGATCTTCCTGGAGGTGCAAGCGATAC
GCGAGACCGTGGAACTGCGTCAGTACGATCCCGTGGCTGCGCTCTTCTTTTTCGATA
TCGACTTGCTGCTGCAGCGCGGGCCTCAGTACAGCGAACACCCCACCTTCACCAGCC
AGTATCGCATCCAGGGCAAGCTTGAGTACCGACACACCTGGGACCGGCACGACGAG
GGTGCCGCCCAGGGCGACGACGACGTCTGGACCAGCGGATCGGACTCCGACGAGGA
ACTCGTAACCACCGAGCGCAAGACGCCCCGCGTTACCGGCGGCGGCGCCATGGCGG
GCGCCTCCACTTCCGCGGGCCGCAAACGCAAATCAGCATCCTCGGCGACGGCGTGC
ACGGCGGGCGTTATGACACGCGGCCGCCTTAAGGCCGAGTCCACCGTCGCGCCCGA
AGAGGACACCGACGAGGATTCCGACAACGAAATCCACAATCCGGCCGTGTTCACCT
GGCCGCCCTGGCAGGCCGGCATCCTGGCCCGCAACCTGGTGCCCATGGTGGCTACG
GTTCAGGGTCAGAATCTGAAGTACCAGGAGTTCTTCTGGGACGCCAACGACATCTAC
CGCATCTTCGCCGAATTGGAAGGCGTATGGCAGCCCGCTGCGCAACCCAAACGTCG
CCGCCACCGGCAAGACGCCTTGCCCGGGCCATGCATCGCCTCGACGCCCAAAAAGC
ACCGAGGTTAG (MMLV Gag nucleotide sequence bolded)
SEQ ID NO: 6 is a Codon Optimized MMLV Gag - CMV pp65
Nucleotide Sequence
(SEQ ID NO: 6)
ATGGGACAGACAGTCACTACACCCCTGAGCCTGACACTGGGACATTGGAAAGA
CGTGGAGAGGATTGCACATAACCAGAGCGTGGACGTGAAGAAACGGAGATGG
GTCACCTTTTGCTCCGCCGAGTGGCCAACATTCAATGTGGGATGGCCCCGAGA
TGGCACCTTCAACCGGGACCTGATCACTCAGGTGAAGATCAAGGTCTTCTCTCC
AGGACCCCACGGCCATCCAGATCAGGTGCCCTACATCGTCACCTGGGAGGCTC
TGGCATTTGACCCCCCTCCATGGGTGAAGCCTTTCGTCCACCCAAAACCACCTC
CACCACTGCCTCCATCTGCCCCTAGTCTGCCACTGGAACCCCCTCGGTCAACCC
CACCCAGAAGCTCCCTGTATCCCGCACTGACACCTAGCCTGGGGGCCAAGCCT
AAACCACAGGTGCTGTCTGATAGTGGCGGGCCTCTGATCGATCTGCTGACCGA
GGACCCTCCACCATACCGCGACCCACGACCTCCACCAAGCGACCGGGACGGAA
ACGGAGGAGAGGCTACACCCGCAGGCGAAGCCCCCGATCCTAGTCCAATGGCA
TCAAGGCTGCGCGGGAGGCGCGAACCTCCAGTGGCCGACTCAACCACAAGCCA
GGCATTTCCACTGAGGGCCGGGGGAAATGGACAGCTCCAGTATTGGCCCTTCT
CTAGTTCAGATCTGTACAACTGGAAGAACAATAACCCTAGCTTCAGCGAGGAC
CCAGGCAAACTGACCGCCCTGATCGAATCCGTGCTGATTACCCACCAGCCCAC
ATGGGACGATTGTCAGCAGCTCCTGGGCACCCTGCTGACCGGAGAGGAAAAGC
AGAGAGTGCTGCTGGAGGCTAGGAAAGCAGTCCGCGGGGACGATGGAAGGCC
AACACAGCTCCCCAATGAGGTGGATGCCGCTTTCCCTCTGGAACGGCCAGATT
GGGACTATACTACCCAGGCTGGACGCAACCACCTGGTGCATTACCGGCAGCTC
CTGCTGGCTGGACTGCAGAATGCAGGGCGCAGCCCCACTAACCTGGCCAAGGT
GAAAGGAATCACCCAGGGCCCCAATGAGTCCCCTTCTGCATTCCTGGAGCGGC
TGAAGGAAGCCTACCGACGGTATACTCCCTACGATCCTGAGGACCCAGGCCAG
GAAACCAACGTGAGTATGAGCTTCATCTGGCAGTCCGCTCCTGACATTGGCCG
AAAACTGGAGCGGCTGGAAGATCTGAAGAACAAGACCCTGGGCGACCTGGTGC
GGGAGGCAGAAAAGATCTTCAACAAAAGGGAGACTCCAGAGGAACGGGAGGA
AAGAATTAGAAGGGAAACAGAGGAAAAGGAGGAACGCCGACGGACTGAGGAT
GAACAGAAGGAGAAAGAAAGAGACCGGCGGCGGCACCGGGAGATGTCTAAGC
TGCTGGCCACCGTGGTCAGTGGCCAGAAACAGGATCGACAGGGAGGAGAGCG
ACGGAGAAGCCAGCTCGATCGGGACCAGTGCGCCTATTGTAAGGAAAAAGGGC
ATTGGGCTAAGGACTGCCCCAAGAAACCCAGAGGCCCACGCGGGCCCCGACCT
CAGACTTCCCTGCTGACCCTGGACGATTGCGAGAGCCGGGGCCGGCGGTGCCCA
GAAATGATCTCTGTGCTGGGGCCCATTAGTGGACATGTGCTGAAGGCCGTCTTCTCC
AGGGGAGACACCCCCGTGCTGCCTCACGAGACTCGACTGCTGCAGACCGGCATCCA
TGTGCGGGTCTCCCAGCCCTCTCTGATTCTGGTGTCACAGTATACACCAGATAGCAC
TCCCTGCCACAGAGGAGACAATCAGCTCCAGGTGCAGCATACCTACTTTACAGGCTC
CGAGGTCGAAAACGTGTCTGTCAATGTGCACAACCCTACCGGCAGGAGCATCTGTC
CTAGCCAGGAGCCAATGAGCATCTACGTGTACGCCCTGCCTCTGAAGATGCTGAATA
TCCCATCAATTAACGTCCACCATTACCCTAGCGCAGCCGAACGGAAGCACAGACAT
CTGCCAGTGGCCGACGCTGTCATCCATGCCAGCGGCAAACAGATGTGGCAGGCAAG
ACTGACCGTGTCCGGGCTGGCCTGGACAAGGCAGCAGAATCAGTGGAAGGAGCCCG
ACGTGTACTATACCAGCGCCTTCGTGTTCCCTACCAAAGACGTGGCCCTGAGACATG
TGGTGTGCGCACATGAGCTGGTGTGCAGCATGGAAAACACTAGGGCCACCAAGATG
CAGGTCATCGGCGATCAGTATGTCAAAGTGTACCTGGAGAGTTTTTGCGAAGACGTG
CCATCAGGGAAGCTGTTCATGCATGTGACCCTGGGCAGCGATGTCGAGGAAGACCT
GACCATGACAAGAAATCCACAGCCCTTTATGAGACCCCACGAGAGGAATGGGTTCA
CTGTGCTGTGCCCCAAGAACATGATCATTAAGCCTGGAAAAATCAGTCATATTATGC
TGGATGTGGCCTTTACATCACACGAGCATTTCGGACTGCTGTGCCCCAAATCCATCC
CTGGACTGAGCATTTCCGGCAATCTGCTGATGAACGGCCAGCAGATCTTCCTGGAAG
TGCAGGCCATCCGGGAGACCGTCGAACTGCGACAGTATGACCCAGTGGCTGCACTG
TTCTTTTTCGACATCGACCTGCTGCTGCAGCGAGGACCACAGTACAGCGAGCACCCT
ACTTTTACCTCCCAGTATCGGATTCAGGGGAAGCTGGAGTACAGGCACACCTGGGAT
CGCCATGACGAAGGAGCCGCTCAGGGGGACGATGACGTGTGGACATCTGGCAGTGA
TTCAGACGAGGAACTGGTGACAACTGAGCGAAAAACCCCCCGGGTGACAGGAGGA
GGGGCAATGGCAGGGGCCAGCACCAGCGCAGGGCGGAAGCGAAAAAGCGCCAGCA
GCGCCACAGCATGTACCGCCGGCGTGATGACTAGAGGAAGGCTGAAGGCCGAGTCT
ACAGTCGCTCCCGAGGAAGATACTGACGAGGATAGTGACAATGAAATCCACAACCC
CGCCGTGTTCACCTGGCCACCTTGGCAGGCAGGGATTCTGGCTCGCAACCTGGTCCC
CATGGTGGCAACCGTCCAGGGACAGAATCTGAAGTATCAGGAGTTTTTCTGGGATGC
TAACGACATCTACCGGATTTTTGCAGAGCTGGAAGGCGTGTGGCAGCCAGCAGCCC
AGCCCAAACGACGGAGACATCGACAGGACGCTCTGCCAGGACCTTGTATCGCCAGC
ACACCAAAGAAGCACAGGGGCTAA (MMLV Gag nucleotide sequence
bolded)
SEQ ID NO: 7 is a Codon Optimized MMLV Gag - CMV pp65
Nucleotide Sequence
(SEQ ID NO: 7)
ATGGGACAGACCGTCACAACACCCCTGAGCCTGACCCTGGGACATTGGAAAGA
CGTGGAGAGGATCGCACATAACCAGAGCGTGGACGTGAAGAAACGGAGATGG
GTCACATTCTGCAGTGCTGAGTGGCCAACTTTTAATGTGGGATGGCCCCGAGA
CGGCACTTTCAACAGGGATCTGATCACCCAGGTGAAGATCAAGGTCTTTAGCC
CAGGACCTCACGGACATCCAGACCAGGTGCCTTATATCGTCACCTGGGAGGCA
CTGGCCTTCGATCCCCCTCCATGGGTGAAGCCATTTGTCCACCCAAAACCACCT
CCACCACTGCCTCCAAGTGCCCCTTCACTGCCACTGGAACCACCCCGGAGCAC
ACCACCCCGCAGCTCCCTGTATCCTGCTCTGACTCCATCTCTGGGCGCAAAGCC
AAAACCACAGGTGCTGAGCGACTCCGGAGGACCACTGATTGACCTGCTGACAG
AGGACCCCCCACCATACCGAGATCCTCGGCCTCCACCAAGCGACCGCGATGGA
AATGGAGGAGAGGCTACTCCTGCCGGCGAAGCCCCTGACCCATCTCCAATGGC
TAGTAGGCTGCGCGGCAGGCGCGAGCCTCCAGTGGCAGATAGCACCACATCCC
AGGCCTTCCCTCTGAGGGCTGGGGGAAATGGGCAGCTCCAGTATTGGCCATTT
TCTAGTTCAGACCTGTACAACTGGAAGAACAATAACCCCTCTTTCAGTGAGGAC
CCCGGCAAACTGACCGCCCTGATCGAATCCGTGCTGATTACCCATCAGCCCAC
ATGGGACGATTGTCAGCAGCTCCTGGGCACCCTGCTGACCGGAGAGGAAAAGC
AGCGCGTGCTGCTGGAGGCTCGCAAAGCAGTCCGAGGGGACGATGGACGGCC
CACACAGCTCCCTAATGAGGTGGACGCCGCTTTTCCACTGGAAAGACCCGACT
GGGATTATACTACCCAGGCAGGGAGAAACCACCTGGTCCATTACAGGCAGCTC
CTGCTGGCAGGCCTGCAGAATGCCGGGAGATCCCCCACCAACCTGGCCAAGGT
GAAAGGCATCACACAGGGGCCTAATGAGTCACCAAGCGCCTTTCTGGAGAGGC
TGAAGGAAGCTTACCGACGGTATACCCCATACGACCCTGAGGACCCCGGACAG
GAAACAAACGTCTCCATGTCTTTCATCTGGCAGTCTGCCCCAGACATTGGGCG
GAAGCTGGAGAGACTGGAAGACCTGAAGAACAAGACCCTGGGCGACCTGGTG
CGGGAGGCTGAAAAGATCTTCAACAAACGGGAGACCCCCGAGGAAAGAGAGG
AAAGGATTAGAAGGGAAACTGAGGAAAAGGAGGAACGCCGACGGACCGAGGA
CGAACAGAAGGAGAAAGAACGAGATCGGCGGCGGCACCGGGAGATGTCAAAG
CTGCTGGCCACCGTGGTCAGCGGACAGAAACAGGACAGACAGGGAGGAGAGC
GACGGAGAAGCCAGCTCGACAGGGATCAGTGCGCATACTGTAAGGAAAAAGGC
CATTGGGCCAAGGATTGCCCCAAAAAGCCAAGAGGACCAAGAGGACCAAGACC
ACAGACATCACTGCTGACCCTGGACGACTGCGAGAGCCGGGGCCGGCGGTGCCC
AGAAATGATCTCTGTGCTGGGGCCCATTAGTGGACATGTGCTGAAGGCCGTCTTCTC
CAGGGGAGACACCCCCGTGCTGCCTCACGAGACTCGACTGCTGCAGACCGGCATCC
ATGTGCGGGTCTCCCAGCCCTCTCTGATTCTGGTGTCACAGTATACACCAGATAGCA
CTCCCTGCCACAGAGGAGACAATCAGCTCCAGGTGCAGCATACCTACTTTACAGGCT
CCGAGGTCGAAAACGTGTCTGTCAATGTGCACAACCCTACCGGCAGGAGCATCTGT
CCTAGCCAGGAGCCAATGAGCATCTACGTGTACGCCCTGCCTCTGAAGATGCTGAAT
ATCCCATCAATTAACGTCCACCATTACCCTAGCGCAGCCGAACGGAAGCACAGACA
TCTGCCAGTGGCCGACGCTGTCATCCATGCCAGCGGCAAACAGATGTGGCAGGCAA
GACTGACCGTGTCCGGGCTGGCCTGGACAAGGCAGCAGAATCAGTGGAAGGAGCCC
GACGTGTACTATACCAGCGCCTTCGTGTTCCCTACCAAAGACGTGGCCCTGAGACAT
GTGGTGTGCGCACATGAGCTGGTGTGCAGCATGGAAAACACTAGGGCCACCAAGAT
GCAGGTCATCGGCGATCAGTATGTCAAAGTGTACCTGGAGAGTTTTTGCGAAGACGT
GCCATCAGGGAAGCTGTTCATGCATGTGACCCTGGGCAGCGATGTCGAGGAAGACC
TGACCATGACAAGAAATCCACAGCCCTTTATGAGACCCCACGAGAGGAATGGGTTC
ACTGTGCTGTGCCCCAAGAACATGATCATTAAGCCTGGAAAAATCAGTCATATTATG
CTGGATGTGGCCTTTACATCACACGAGCATTTCGGACTGCTGTGCCCCAAATCCATC
CCTGGACTGAGCATTTCCGGCAATCTGCTGATGAACGGCCAGCAGATCTTCCTGGAA
GTGCAGGCCATCCGGGAGACCGTCGAACTGCGACAGTATGACCCAGTGGCTGCACT
GTTCTTTTTCGACATCGACCTGCTGCTGCAGCGAGGACCACAGTACAGCGAGCACCC
TACTTTTACCTCCCAGTATCGGATTCAGGGGAAGCTGGAGTACAGGCACACCTGGGA
TCGCCATGACGAAGGAGCCGCTCAGGGGGACGATGACGTGTGGACATCTGGCAGTG
ATTCAGACGAGGAACTGGTGACAACTGAGCGAAAAACCCCCCGGGTGACAGGAGG
AGGGGCAATGGCAGGGGCCAGCACCAGCGCAGGGCGGAAGCGAAAAAGCGCCAGC
AGCGCCACAGCATGTACCGCCGGCGTGATGACTAGAGGAAGGCTGAAGGCCGAGTC
TACAGTCGCTCCCGAGGAAGATACTGACGAGGATAGTGACAATGAAATCCACAACC
CCGCCGTGTTCACCTGGCCACCTTGGCAGGCAGGGATTCTGGCTCGCAACCTGGTCC
CCATGGTGGCAACCGTCCAGGGACAGAATCTGAAGTATCAGGAGTTTTTCTGGGATG
CTAACGACATCTACCGGATTTTTGCAGAGCTGGAAGGCGTGTGGCAGCCAGCAGCC
CAGCCCAAACGACGGAGACATCGACAGGACGCTCTGCCAGGACCTTGTATCGCCAG
CACACCAAAGAAGCACAGGGGCTAA (MMLV Gag nucleotide sequence
bolded)
SEQ ID NO: 8 is a HCMV gB Amino Acid Sequence
(SEQ ID NO: 8)
MESRIWCLVVCVNLCIVCLGAAVSSSSTRGTSATHSEIHSSHTTSAAHSRSGSVSQRVTSS
QTVSHGVNETIYNTTLKYGDVVGVNTTKYPYRVCSMAQGTDLIRFERNIVCTSMKPINE
DLDEGIMVVYKRNIVAHTFKVRVYQKVLTFRRSYAYIHTTYLLGSNTEYVAPPMWEIH
HINSHSQCYS SYSRVIAGTVFVAYHRDSYENKTMQLMPDDYSNTHSTRYVTVKDQWHS
RGSTWLYRETCNLNCMVTITTARSKYPYHFFATSTGDVVDISPFYNGTNRNASYFGENA
DKFFIFPNYTIVSDFGRPNSALETHRLVAFLERADSVISWDIQDEKNVTCQLTFWEASERT
IRSEAEDSYHFSSAKMTATFLSKKQEVNMSDSALDCVRDEAINKLQQIFNTSYNQTYEK
YGNVSVFETTGGLVVFWQGIKQKSLVELERLANRSSLNLTHNRTKRSTDGNNATHLSN
MESVHNLVYAQLQFTYDTLRGYINRALAQIAEAWCVDQRRTLEVFKELSKINPSAILSAI
YNKPIAARFMGDVLGLASCVTINQTSVKVLRDMNVKESPGRCYSRPVVIFNFANSSYVQ
YGQLGEDNEILLGNHRTEECQLPSLKIFIAGNSAYEYVDYLFKRMIDLSSISTVDSMIALD
IDPLENTDFRVLELYSQKELRSINVFDLEEIMREFNSYKQRVKYVEDKVVDPLPPYLKGL
DDLMSGLGAAGKAVGVAIGAVGGAVASVVEGVATFLKNPFGAFTIILVAIAVVIIIYLIY
TRQRRLCMQPLQNLFPYLVSADGTTVTSGNTKDTSLQAPPSYEESVYNSGRKGPGPPSS
DASTAAPPYTNEQAYQMLLALVRLDAEQRAQQNGTDSLDGQTGTQDKGQKPNLLDRL
RHRKNGYRHLKDSDEEENV* (TM and CD underlined)
SEQ ID NO: 9 is a HCMV gB Nucleotide Sequence
(SEQ ID NO: 9)
ATGGAGTCAAGGATTTGGTGCCTGGTCGTGTGCGTCAATCTGTGCATCGTCTGTCTG
GGGGCTGCCGTGTCATCAAGTTCTACAAGAGGCACCAGCGCCACCCACTCACACCA
TAGCTCCCATACCACATCCGCCGCTCACTCCCGGTCTGGCAGCGTGAGCCAGAGAGT
CACATCTAGTCAGACCGTGAGCCACGGGGTCAACGAGACCATCTACAATACTACCC
TGAAGTATGGCGACGTGGTCGGGGTGAACACAACTAAATACCCATATAGGGTCTGC
AGTATGGCCCAGGGCACTGATCTGATTAGATTCGAAAGGAACATCGTGTGCACCAG
CATGAAGCCCATTAATGAGGACCTGGATGAAGGGATCATGGTGGTCTACAAACGCA
ATATTGTGGCCCATACCTTCAAGGTGCGAGTCTATCAGAAAGTGCTGACATTTCGGA
GATCTTACGCATATATCCACACCACATACCTGCTGGGGAGTAACACCGAGTATGTGG
CTCCCCCTATGTGGGAAATTCACCATATCAATAGCCATTCCCAGTGCTACTCAAGCT
ACAGCAGAGTGATCGCTGGAACAGTGTTCGTCGCATACCACAGAGACTCTTATGAG
AACAAGACTATGCAGCTCATGCCCGACGATTACAGCAATACACATTCCACTAGATAT
GTGACAGTCAAAGATCAGTGGCACTCAAGGGGCAGCACCTGGCTGTACCGCGAGAC
ATGCAACCTGAATTGTATGGTGACTATCACTACCGCTAGATCCAAGTACCCCTATCA
CTTCTTTGCAACTTCCACCGGGGACGTGGTCGATATTTCTCCTTTCTACAACGGCACA
AACCGGAATGCATCTTATTTTGGGGAGAACGCCGACAAGTTCTTTATTTTCCCAAAT
TACACCATCGTGTCTGATTTTGGCAGACCCAACAGTGCCCTGGAGACACATCGACTG
GTGGCATTCCTGGAACGGGCCGACTCCGTCATTTCTTGGGACATCCAGGATGAGAAG
AATGTGACCTGCCAGCTCACCTTCTGGGAGGCCAGCGAACGCACCATCCGATCCGA
GGCTGAAGATTCTTACCACTTCTCCTCTGCCAAAATGACAGCTACTTTTCTGAGCAA
GAAACAGGAGGTGAACATGTCTGACAGTGCTCTGGATTGCGTGCGGGACGAAGCAA
TTAATAAGCTGCAGCAGATCTTCAACACATCATACAACCAGACTTACGAGAAGTAC
GGAAACGTGAGCGTCTTCGAAACAACTGGCGGGCTGGTGGTCTTTTGGCAGGGCAT
CAAGCAGAAATCCCTGGTGGAGCTGGAAAGGCTGGCCAATCGCAGTTCACTGAACC
TGACTCATAATCGGACCAAGAGATCTACAGACGGAAACAATGCCACACATCTGTCT
AACATGGAGAGTGTGCACAATCTGGTCTACGCTCAGCTCCAGTTTACCTACGACACA
CTGAGAGGCTATATTAACAGGGCACTGGCCCAGATCGCTGAAGCATGGTGCGTGGA
TCAGAGGCGCACCCTGGAGGTCTTCAAGGAACTGTCCAAAATCAACCCTTCAGCAA
TTCTGAGCGCCATCTACAATAAGCCAATTGCAGCCAGGTTTATGGGAGACGTGCTGG
GCCTGGCCAGTTGCGTCACTATCAACCAGACCTCAGTGAAGGTCCTGCGCGATATGA
ATGTGAAAGAGAGTCCCGGCAGATGCTATTCACGGCCTGTGGTCATCTTCAACTTTG
CTAATAGCTCCTACGTGCAGTATGGACAGCTCGGCGAGGACAACGAAATTCTGCTG
GGGAATCACAGGACCGAGGAATGTCAGCTCCCTAGCCTGAAGATTTTCATCGCTGG
AAACTCCGCATACGAGTATGTGGATTACCTGTTCAAGCGGATGATTGACCTGTCTAG
TATCTCCACTGTGGATTCTATGATTGCCCTGGACATCGATCCACTGGAAAATACCGA
CTTCAGGGTGCTGGAGCTGTATAGCCAGAAGGAACTGCGCTCCATCAACGTGTTCGA
TCTGGAGGAAATTATGAGAGAGTTTAATAGCTACAAGCAGAGGGTGAAATATGTCG
AAGATAAGGTGGTCGACCCCCTGCCACCCTACCTGAAAGGCCTGGACGATCTGATG
AGCGGGCTGGGAGCTGCAGGGAAGGCAGTGGGAGTCGCTATCGGCGCAGTGGGAG
GAGCCGTGGCCAGCGTGGTCGAGGGAGTGGCAACATTCCTGAAAAACCCCTTCGGG
GCCTTCACCATCATTCTGGTGGCAATCGCCGTGGTCATCATTATCTACCTGATCTACA
CAAGGCAGCGGCGGCTGTGCATGCAGCCTCTGCAGAACCTGTTTCCATACCTGGTGA
GCGCCGACGGGACCACAGTCACCTCAGGAAATACTAAGGATACCTCTCTGCAGGCC
CCCCCAAGTTACGAGGAATCAGTGTATAACAGCGGCAGAAAAGGACCAGGACCACC
TTCAAGCGACGCCAGCACTGCCGCTCCACCCTACACCAATGAGCAGGCCTATCAGAT
GCTGCTGGCTCTGGTGCGCCTGGATGCCGAACAGCGAGCTCAGCAGAACGGGACCG
ACTCCCTGGATGGACAGACCGGAACACAGGACAAGGGACAGAAACCTAATCTGCTG
GATCGGCTGCGGCACAGAAAAAACGGGTATAGGCACCTGAAGGACTCCGACGAAG
AAGAAAATGTCTAA (TM and CD underlined)
SEQ ID NO: 10 is a Codon Optimized HCMV gB Nucleotide
Sequence
(SEQ ID NO: 10)
ATGGAATCCAGGATCTGGTGCCTGGTAGTCTGCGTTAACTTGTGTATCGTCTGTCTG
GGTGCTGCGGTTTCCTCATCTTCTACTCGTGGAACTTCTGCTACTCACAGTCACCATT
CCTCTCATACGACGTCTGCTGCTCATTCTCGATCCGGTTCAGTCTCTCAACGCGTAAC
TTCTTCCCAAACGGTCAGCCATGGTGTTAACGAGACCATCTACAACACTACCCTCAA
GTACGGAGATGTGGTGGGGGTCAACACCACCAAGTACCCCTATCGCGTGTGTTCTAT
GGCACAGGGTACGGATCTTATTCGCTTTGAACGTAATATCGTCTGCACCTCGATGAA
GCCCATCAATGAAGACCTGGACGAGGGCATCATGGTGGTCTACAAACGCAACATCG
TCGCGCACACCTTTAAGGTACGAGTCTACCAGAAGGTTTTGACGTTTCGTCGTAGCT
ACGCTTACATCCACACCACTTATCTGCTGGGCAGCAACACGGAATACGTGGCGCCTC
CTATGTGGGAGATTCATCATATCAACAGTCACAGTCAGTGCTACAGTTCCTACAGCC
GCGTTATAGCAGGCACGGTTTTCGTGGCTTATCATAGGGACAGCTATGAAAACAAA
ACCATGCAATTAATGCCCGACGATTATTCCAACACCCACAGTACCCGTTACGTGACG
GTCAAGGATCAATGGCACAGCCGCGGCAGCACCTGGCTCTATCGTGAGACCTGTAA
TCTGAATTGTATGGTGACCATCACTACTGCGCGCTCCAAGTATCCCTATCATTTTTTC
GCAACTTCCACGGGTGATGTGGTTGACATTTCTCCTTTCTACAACGGAACTAATCGC
AATGCCAGCTATTTTGGAGAAAACGCCGACAAGTTTTTCATTTTTCCGAACTACACT
ATCGTCTCCGACTTTGGAAGACCGAATTCTGCGTTAGAGACCCACAGGTTGGTGGCT
TTTCTTGAACGTGCGGACTCAGTGATCTCCTGGGATATACAGGACGAGAAGAATGTT
ACTTGTCAACTCACTTTCTGGGAAGCCTCGGAACGCACCATTCGTTCCGAAGCCGAG
GACTCGTATCACTTTTCTTCTGCCAAAATGACCGCCACTTTCTTATCTAAGAAGCAAG
AGGTGAACATGTCCGACTCTGCGCTGGACTGTGTACGTGATGAGGCCATAAATAAGT
TACAGCAGATTTTCAATACTTCATACAATCAAACATATGAAAAATATGGAAACGTGT
CCGTCTTTGAAACCACTGGTGGTTTGGTGGTGTTCTGGCAAGGTATCAAGCAAAAAT
CTCTGGTGGAACTCGAACGTTTGGCCAACCGCTCCAGTCTGAATCTTACTCATAATA
GAACCAAAAGAAGTACAGATGGCAACAATGCAACTCATTTATCCAACATGGAGTCG
GTGCACAATCTGGTCTACGCCCAGCTGCAGTTCACCTATGACACGTTGCGCGGTTAC
ATCAACCGGGCGCTGGCGCAAATCGCAGAAGCCTGGTGTGTGGATCAACGGCGCAC
CCTAGAGGTCTTCAAGGAACTTAGCAAGATCAACCCGTCAGCTATTCTCTCGGCCAT
CTACAACAAACCGATTGCCGCGCGTTTCATGGGTGATGTCCTGGGTCTGGCCAGCTG
CGTGACCATTAACCAAACCAGCGTCAAGGTGCTGCGTGATATGAATGTGAAGGAAT
CGCCAGGACGCTGCTACTCACGACCAGTGGTCATCTTTAATTTCGCCAACAGCTCGT
ACGTGCAGTACGGTCAACTGGGCGAGGATAACGAAATCCTGTTGGGCAACCACCGC
ACTGAGGAATGTCAGCTTCCCAGCCTCAAGATCTTCATCGCCGGCAACTCGGCCTAC
GAGTACGTGGACTACCTCTTCAAACGCATGATTGACCTCAGCAGCATCTCCACCGTC
GACAGCATGATCGCCCTAGACATCGACCCGCTGGAAAACACCGACTTCAGGGTACT
GGAACTTTACTCGCAGAAAGAATTGCGTTCCATCAACGTTTTTGATCTCGAGGAGAT
CATGCGCGAGTTCAATTCGTATAAGCAGCGGGTAAAGTACGTGGAGGACAAGGTAG
TCGACCCGCTGCCGCCCTACCTCAAGGGTCTGGACGACCTCATGAGCGGCCTGGGCG
CCGCGGGAAAGGCCGTTGGCGTAGCCATTGGGGCCGTGGGTGGCGCGGTGGCCTCC
GTGGTCGAAGGCGTTGCCACCTTCCTCAAAAACCCCTTCGGAGCCTTCACCATCATC
CTCGTGGCCATAGCCGTCGTCATTATCATTTATTTGATCTATACTCGACAGCGGCGTC
TCTGCATGCAGCCGCTGCAGAACCTCTTTCCCTATCTGGTGTCCGCCGACGGGACCA
CCGTGACGTCGGGCAACACCAAAGACACGTCGTTACAGGCTCCGCCTTCCTACGAG
GAAAGTGTTTATAATTCTGGTCGCAAAGGACCGGGACCACCGTCGTCTGATGCATCC
ACGGCGGCTCCGCCTTACACCAACGAGCAGGCTTACCAGATGCTTCTGGCCCTGGTC
CGTCTGGACGCAGAGCAGCGAGCGCAGCAGAACGGTACAGATTCTTTGGACGGACA
GACTGGCACGCAGGACAAGGGACAGAAGCCCAACCTGCTAGACCGACTGCGACACC
GCAAAAACGGCTACCGACACTTGAAAGACTCCGACGAAGAAGAGAACGTCTGA
(TM and CD underlined)

### DETAILED DESCRIPTION OF THE EMBODIMENTS

GBM responds poorly to treatment due to a number of factors including the localization of the tumour, the inherent resistance of the cells to chemotherapy, and brain cells' poor capacity for self-repair. Typically, GBM tumours are surgically removed to the extent possible; however, complete removal is usually impossible due to the rapid invasion of GBM cells into surrounding tissue. Radiation and chemotherapy are often used following surgical treatment in an attempt to delay progression of the disease. However, GBM tumours usually recur and median survival time in treated patients is only between twelve and fifteen months.

In recent years, immunotherapies have been proposed as treatments for GBM, based on the knowledge that T cells have been shown to kill tumour cells and infiltrate brain tumours. However, the development of immunotherapeutic agents to treat GBM has proven challenging because of the diversity of the tumour cells and the lack of a common tumour rejection antigen which could act as an immune target. As well, many GBM patients demonstrate a variety of different T-cell dysfunction including anergy, tolerance and T-cell exhaustion (Woroniecka et al, Clin Cancer Res. (2018) 24 4175-4186). They also can show a weakened antibody response.

Several anti-cancer immunotherapies have been developed which are directed to regulating immune checkpoints, specifically the molecules that simulate or inhibit the activity of immune cells. For example, regulators such as PD-1 and PD-L1 are known to inhibit the activity of T cells and therefore they have become attractive targets for immunotherapeutic drugs, which have been used successfully to treat different forms of cancer. Some survival benefit was observed in a small study of GBM patients treated with an anti-PD1 inhibitor (Cloughesy et al., Nature Medicine, (2019) 25: 477-486); however, a larger phase 3 study failed when an anti-PD1 inhibitor in combination with radiation failed to extend the lives of patients when compared to chemotherapy with radiation (BMS—Optivo CheckMate 498 Clinical Trial—May 9, 2019).

Other studies have attempted vaccination with synthetic peptides that lower the risk of autoimmunity (Schuster Neuro Oncol 2015, 17:854-861). EGFRvIII is a truncated variant of epidermal growth factor receptor (“EGFR”) that is found in about 30% of GBM, but not in normal cells. Early Phase I and II clinical trials using vaccination against a 13-mer peptide from EGFRvIII demonstrated significant increased overall survival to 26 months in immunized patients with recurrent GBMs, providing encouraging support for therapeutic vaccination against GBMs. However, a larger Phase III study in newly diagnosed GBM patients was halted after the drug showed no survival benefit (ABBVIE press release, May 17, 2019). In addition to the disappointing Phase III results, EGFRvIII vaccination is limited to only a subset of GBM patients whose tumors express EGFRvIII, and immune escape of tumor cells lacking the EGFRvIII antigen after vaccination is already evident, limiting the long-term efficacy of this approach (Swampson, J. H. J of Clinical Oncol. 2010; 28:4722-4729).

Other immunotherapeutic approaches to treat GBM have been proposed based on the discovery of viral antigens in GBM tumour cells, which show lower expression in normal brain tissue. As early as 2002, it was discovered that human cytomegalovirus (HCMV) was present in GBM cells (Cobbs et al, Can. Res (2002) 62:3347). HCMV, a β-herpesvirus, is a ubiquitously occurring pathogen. In an immunocompetent person, HCMV infection is normally unnoticed, having at most mild and nonspecific symptoms. HCMV DNA and proteins are expressed in over 90% of GBM cells but not in the surrounding normal brain tissue (Dziurzynski et al, Neuro-Onc. (2012) 14:246). Although the role of HCMV in GBM is not well understood, the HCMV glycoprotein B (gB) has been shown to mediate glioma cell entry by binding to the receptor tyrosine kinase PDGFR-alpha (PDGFRα), resulting in activation of the PI3 kinase/Akt signaling pathway, which enhances both tumor cell growth and invasiveness (Cobbs, C., Oncotarget 2014; 5:1091-1100). Low levels of HCMV expression have been correlated with improved overall survival in GBM patients (Rahbar, A. Herpesviridae 2012; 3:3).

The ubiquitous presence of HCMV in GBM cells has led to suggestions that HCMV antigens could constitute therapeutic targets for immunotherapeutic treatment. Of particular potential benefit to the use of HCMV antigens as targets is that they are recognized immunologically as being “foreign,” and T cells have a much higher affinity for foreign antigens than for self-antigens.

Some studies have investigated immunotherapy directed against HCMV antigens in the treatment of GBM. In one study, HCMV-specific T cells (CD4+ and CD8+ polyfunctional T cells) were shown to recognize and kill autologous GBM tumor cells, providing evidence that HCMV antigens are presented by tumor cells at immunologically relevant levels (Nair, SK., Clin Cancer Res 2014; 20: 2684-2694). Extending these observations into the clinic, adoptive T cell therapy with autologous HCMV-specific T cells demonstrated encouraging early clinical results, with 4 out of 10 patients remaining disease free during the study period (Schuessler, A. Cancer Res. 2014; 74: 3466-3476).

While these preliminary studies showed promise for HCMV-targeted immunotherapies, other studies showed that GBM patients show a significantly lower immune response to HCMV compared to healthy persons (Liu et al, J. Trans Med., 2018 16: 182). In particular, GBM patients were shown to produce significantly lower anti-HCMV antibodies (IgG) compared to healthy subjects who are HCMV positive (Liu, 2018). In one study, 31% of patients with GBM tumors that had HCMV completely lacked anti-CMV antibodies (Rahbar, 2015). Accordingly, many GBM patients have significant dysregulation of immunity against HCMV, which creates challenges in developing immunotherapeutic treatments based on HCMV antigens.

In order to overcome weakened immunity to HCMV, researchers have developed a treatment using dendritic cells from GBM patients which are pulsed with RNA for an HCMV antigen. A small, controlled phase I trial demonstrated that dendritic cell preconditioning at the injection site two days prior to vaccination with autologous dendritic cells pulsed with RNA for the HCMV non-structural protein, pp65, significantly improved overall survival in patients with primary GBM (Mitchell, D. A. Nature 2015; 519: 366-369). The substantial increase of overall survival observed in patients was correlated with high serum levels of CCL3, a chemokine associated with dendritic cell mobilization, and this biomarker was confirmed in mouse models. More recently, the same HCMV pp65 dendritic cell vaccine in combination with temozolomide chemotherapy improved the survival time of GBM patients (Batich et al, (2017) Clin Cancer Res 23 1898-1909). In this study, IFN-γ-secreting CD8+ T cells against the HCMV pp65 antigen correlated with clinical benefit. While these studies demonstrate that the HCMV pp65 protein can constitute a potential target for immunotherapy, this method requires that unique pulsed dendritic cells be produced for each patient, entailing a level of personalized treatment which is both costly and unavailable in most populations.

A need exists for an accessible immunotherapeutic treatment for GBM, which effectively targets GBM tumour cells but can be formulated for use in a broad patient population.

The present disclosure provides immunotherapeutic compositions and methods of their use for treatment of GBM. The immunogenic compositions of the invention stimulate anti-HCMV T cell immunity against HCMV-expressing GBM tumours. In addition, the compositions of the disclosure have demonstrated clinical efficacy, in terms of tumour response and improved survival time in GBM patients. In particular, clinical subjects who responded to the immunogenic compositions of the invention demonstrated a 6.25 month improvement in median overall survival time compared to those subjects that didn't respond to the treatment. Unexpectedly, at doses of at least 10 μg pp65 and 200 μg GM-CSF, the compositions of the invention were able to induce a response in GBM patients who had demonstrated significant immune dysregulation against HCMV, as shown by a lack of antibody response to the HCMV gB antigen.

The immunotherapeutic compositions of the disclosure comprise virus-like particles (“VLPs”). VLPs are multiprotein structures which are generally composed of one or more viral proteins. VLPs mimic the conformation of viruses but lack genetic material, and therefore are not infectious. They can form (or “self-assemble”) upon expression of a viral structural protein under appropriate circumstances. VLPs overcome some of the disadvantages of vaccines prepared using attenuated viruses because they can be produced without the need to have any live virus present during the production process. A wide variety of VLPs have been prepared. For example, VLPs including single or multiple capsid proteins either with or without envelope proteins and/or surface glycoproteins have been prepared. In some cases, VLPs are non-enveloped and assemble by expression of just one major capsid protein. In other cases, VLPs are enveloped and can comprise multiple antigenic proteins found in the corresponding native virus. Self-assembly of enveloped VLPs is more complex than non-enveloped VLPs because of the complex reactions required for fusion with the host cell membrane (Garrone et al., 2011 Science Trans. Med. 3: 1-8) and “budding” of the VLP to form a fully enveloped separate particle. Formation of intact VLPs can be confirmed by imaging of the particles using electron microscopy.

VLPs typically resemble their corresponding native virus and can be multivalent particulate structures. Presentation of surface glycoproteins in the context of a VLP is advantageous for induction of neutralizing antibodies against the polypeptide as compared to other forms of antigen presentation, e.g., soluble antigens not associated with a VLP. Neutralizing antibodies most often recognize tertiary or quaternary structures; this often requires presenting antigenic proteins, like envelope glycoproteins, in their native viral conformation.

Antigens expressed on the surface of the VLPs can also induce a CD4-restricted T helper cell response that can help elicit and sustain both neutralizing antibody and cytotoxic T lymphocyte (CTL) responses. In contrast, antigens expressed within the internal space of the VLP may promote CD8-restricted CTL responses through dendritic cell uptake of VLPs in a process referred to as cross-priming and presentation.

The VLPs of the disclosure can comprise a retroviral vector. Retroviruses are enveloped RNA viruses that belong to the family Retroviridae. After infection of a host cell by a retrovirus, RNA is transcribed into DNA via the enzyme reverse transcriptase. DNA is then incorporated into the host cell's genome by an integrase enzyme and thereafter replicates as part of the host cell's DNA. The Retroviridae family includes the following genera Alpharetrovirus, Betaretrovirus, Gammearetrovirus, Deltaretrovirus, Epsilonretrovirus, Lentivirus and Spumavirus. The hosts for this family of retroviruses generally are vertebrates. Retroviruses produce an infectious virion containing a spherical nucleocapsid (the viral genome in complex with viral structural proteins) surrounded by a lipid bilayer derived from the host cell membrane.

Retroviral vectors can be used to generate VLPs that lack a retrovirus-derived genome and are therefore non-replicating. This is accomplished by replacement of most of the coding regions of the retrovirus with genes or nucleotide sequences to be transferred; so that the vector is incapable of making proteins required for additional rounds of replication. Depending on the properties of the glycoproteins present on the surface of the particles, VLPs have limited ability to bind to and enter the host cell but cannot propagate. Therefore, VLPs can be administered safely as an immunogenic composition.

The present invention utilizes VLPs comprising a retroviral structural protein, Murine Leukemia Virus (MLV) structural protein and, in particular, a Moloney Murine Leukemia Virus (MMLV). Genomes of these retroviruses are readily available in databases.

The retroviral structural protein for use in accordance with the present invention is a Gag polypeptide. The Gag proteins of retroviruses have an overall structural similarity and, within each group of retroviruses, are conserved at the amino acid level. Retroviral Gag proteins primarily function in viral assembly. Expression of Gag of some viruses (e.g., murine leukemia viruses, such as MMLV) in some host cells, can result in self-assembly of the expression product into VLPs. The Gag gene expression product in the form of a polyprotein gives rise to the core structural proteins of the VLP. Functionally, the Gag polyprotein is divided into three domains: the membrane binding domain, which targets the Gag polyprotein to the cellular membrane, the interaction domain which promotes Gag polymerization and the late domain which facilitates release of nascent virions from the host cell. In general, the form of the Gag protein that mediates viral particle assembly is the polyprotein. Retroviruses assemble an immature capsid composed of the Gag polyprotein but devoid of other viral elements like viral protease with Gag as the structural protein of the immature virus particle.

The MMLV Gag gene encodes a 65 kDa polyprotein precursor which is proteolytically cleaved into 4 structural proteins (Matrix (MA); p12; Capsid (CA); and Nucleocapsid (NC)), by MLV protease, in the mature virion. In the absence of MLV protease, the polyprotein remains uncleaved, and the resulting particle remains in an immature form. The gene encoding the MMLV nucleic acid is provided herein as SEQ ID NO: 2. An exemplary codon optimized sequence of MMLV nucleic acid is provided as SEQ ID NO: 3.

Therefore, in some embodiments, a Gag polypeptide suitable for the present invention is substantially homologous to an MMLV Gag polypeptide which is SEQ ID NO:1. In some embodiments, a Gag polypeptide suitable for the present invention has an amino acid sequence at least 80%, 85%, 90%, 91%, 92%, 93%, 94%, 95%, 96%, 97%, 98%, 99% or more homologous to SEQ ID NO:1. In some embodiments, a Gag polypeptide suitable for the present invention is substantially identical to, or identical to SEQ ID NO: 1 or a codon degenerate version thereof. Gag polypeptide variants sharing at least 80%, 85%, 90%, 91%, 92%, 93%, 94%, 95%, 96%, 97%, 98%, 99% or more identity to SEQ ID NO:1 are known in the art.

In some embodiments, a suitable MMLV Gag polypeptide is encoded by a nucleic acid sequence having at least 80%, 85%, 90%, 91%, 92%, 93%, 94%, 95%, 96%, 97%, 98%, 99% sequence identity to SEQ ID NO:2. In some embodiments, a suitable MMLV Gag polypeptide is encoded by a nucleic acid sequence having SEQ ID NO: 2 or a codon degenerate version thereof.

As is well known to those of skill in the art, it is possible to improve the expression of a nucleic acid sequence in a host organism by replacing the nucleic acids coding for a particular amino acid (i.e. a codon) with another codon which is better expressed in the host organism. One reason that this effect arises is due to the fact that different organisms show preferences for different codons. The process of altering a nucleic acid sequence to achieve better expression based on codon preference is called codon optimization. Various methods are known in the art to analyze codon use bias in various organisms and many computer algorithms have been developed to implement these analyses in the design of codon optimized gene sequences. Therefore, in some embodiments, a suitable MMLV Gag polypeptide is encoded by a codon optimized version of a nucleic acid sequence encoding MMLV Gag and having at least 80%, 85%, 90%, 91%, 92%, 93%, 94%, 95%, 96%, 97%, 98%, 99% sequence identity to SEQ ID NO:3. In some embodiments, a suitable MMLV-Gag polypeptide is encoded by a nucleic acid sequence which is substantially identical to, or identical to, SEQ ID NO: 3.

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Examples of such programs are described in Altschul, et al., 1990, J. Mol. Biol., 215(3): 403-410; Altschul, et al., 1996, Methods in Enzymology 266:460-480; Altschul, et al., 1997 Nucleic Acids Res. 25:3389-3402; Baxevanis, et al., 1998, Bioinformatics: A Practical Guide to the Analysis of Genes and Proteins, Wiley; and Misener, et al., (eds.), 1999, Bioinformatics Methods and Protocols (Methods in Molecular Biology, Vol. 132), Humana Press. In addition to identifying homologous sequences, the programs mentioned above typically provide an indication of the degree of homology. In some embodiments, two sequences are considered to be substantially homologous if at least 50%, 55%, 60%, 65%, 70%, 75%, 80%, 85%, 90%, 91%, 92%, 93%, 94%, 95%, 96%, 97%, 98%, 99% or more of their corresponding residues are homologous over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

The Gag polypeptide used in the invention may be a modified retroviral Gag polypeptide containing one or more amino acid substitutions, deletions, and/or insertions as compared to a wild-type or naturally-occurring Gag polypeptide while retaining substantial self-assembly activity. Typically, in nature, a Gag protein includes a large C-terminal extension which may contain retroviral protease, reverse transcriptase, and integrase enzymatic activity. Assembly of VLPs, however, generally does not require the presence of such components. In some cases, a retroviral Gag protein alone (e.g., lacking a C-terminal extension, lacking one or more of genomic RNA, reverse transcriptase, viral protease, or envelope protein) can self-assemble to form VLPs both in vitro and in vivo (Sharma S et al., 1997, Proc. Natl. Acad. Sci. USA 94: 10803-8).

The Gag polypeptide for use in accordance with the present invention lacks a C-terminal extension and is expressed as a fusion protein with the pp65 antigen from HCMV. In naturally occurring HCMV, pp65 is located within the tegument between the capsid and the viral envelope. It is a major target of the cytotoxic T-cell response and is known to stimulate formation of T-helper cells and also induce cytotoxic T lymphocytes (CTL) against HCMV. The pp65 polypeptide is spliced in frame into the Gag polypeptide coding sequence, e.g., at the 3′ end of the Gag polypeptide coding sequence. The Gag polypeptide coding sequence and the pp65 antigen are expressed by a single promoter.

The VLPs of the invention also express the HCMV gB envelope glycoprotein on the surface of the VLP. gB is one of the major B-cell antigens in HCMV, inducing neutralizing, protective immune responses including potent humoral immune responses. In some embodiments, the immunogenic compositions of the present invention comprise a VLP comprising a wild type envelope HCMV gB polypeptide, the sequence of which is SEQ ID NO: 8 or a codon degenerate version of SEQ ID NO. 8. A nucleic acid which encodes for the polypeptide is shown as SEQ ID NO: 9. A codon optimized version of SEQ ID NO: 9 is shown as SEQ ID NO: 10. In some embodiments, an immunogenic composition of the invention comprises a VLP comprising a gB polypeptide having an amino acid sequence which is at least 80%, 85%, 90%, 91%, 92%, 93%, 94%, 95%, 96%, 97%, 98%, 99% or more homologous to SEQ ID NO: 8. In some embodiments, the polypeptide is encoded by a nucleic acid sequence at least 80%, 85%, 90%, 91%, 92%, 93%, 94%, 95%, 96%, 97%, 98%, 99% or more homologous to SEQ ID NO: 9. In some embodiments, the polypeptide is encoded by a codon optimized version of the nucleic acid sequence of SEQ ID NO: 9, which is at least 80%, 85%, 90%, 91%, 92%, 93%, 94%, 95%, 96%, 97%, 98%, 99% or more homologous to the SEQ ID NO: 10.

It will be appreciated that a composition comprising VLPs will typically include a mixture of VLPs with a range of sizes. It is to be understood that the diameter values listed below correspond to the most frequent diameter within the mixture. In some embodiments >90% of the vesicles in a composition will have a diameter which lies within 50% of the most frequent value (e.g., 1000±500 nm). In some embodiments the distribution may be narrower, e.g., >90% of the vesicles in a composition may have a diameter which lies within 40, 30, 20, 10 or 5% of the most frequent value. In some embodiments, sonication or ultra-sonication may be used to facilitate VLP formation and/or to alter VLP size. In some embodiments, filtration, dialysis and/or centrifugation may be used to adjust the VLP size distribution.

In general, VLPs of the present disclosure may be of any size. In certain embodiments, the composition may include VLPs with diameters in the range of about 20 nm to about 300 nm. In some embodiments, a VLP is characterized in that it has a diameter within a range bounded by a lower limit of 20, 30, 40, 50, 60, 70, 80, 90, or 100 nm and bounded by an upper limit of 300, 290, 280, 270, 260, 250, 240, 230, 220, 210, 200, 190, 180, or 170 nm. In some embodiments, VLPs within a population show an average diameter within a range bounded by a lower limit of 20, 30, 40, 50, 60, 70, 80, 90, or 100 nm and bounded by an upper limit of 300, 290, 280, 270, 260, 250, 240, 230, 220, 210, 200, 190, 180, or 170 nm. In some embodiments, VLPs in a population have a polydispersity index that is less than 0.5 (e.g., less than 0.45, less than 0.4, or less than 0.3). In some embodiments, VLP diameter is determined by nanosizing. In some embodiments, VLP diameter is determined by electron microscopy.

VLPs in accordance with the present invention may be prepared according to general methodologies known to the skilled person. For example, nucleic acid molecules, reconstituted vectors or plasmids may be prepared using techniques well known to the skilled artisan. Recombinant expression of the polypeptides for VLPs requires construction of an expression vector containing a polynucleotide that encodes one or more polypeptide(s). Once a polynucleotide encoding one or more polypeptides has been obtained, the vector for production of the polypeptide may be produced by recombinant DNA technology using techniques known in the art. Expression vectors that may be utilized in accordance with the present invention include, but are not limited to mammalian and avian expression vectors, bacculovirus expression vectors, plant expression vectors (e.g., Cauliflower Mosaic Virus (CaMV), Tobacco Mosaic Virus (TMV)), plasmid expression vectors (e.g., Ti plasmid), among others.

The VLPs of the invention may be produced in any available protein expression system. Typically, the expression vector is transferred to a host cell by conventional techniques and the transfected cells are then cultured by conventional techniques to produce VLPs. In some embodiments, VLPs are produced using transient transfection of cells. In some embodiments, VLPs are produced using stably transfected cells. Typical cell lines that may be utilized for VLP production include, but are not limited to, mammalian cell lines such as human embryonic kidney (HEK) 293, WI 38, Chinese hamster ovary (CHO), monkey kidney (COS), HT1080, C10, HeLa, baby hamster kidney (BHK), 3T3, C127, CV-1, HaK, NS/O, and L-929 cells. Specific non-limiting examples include, but are not limited to, BALB/c mouse myeloma line (NSO/l, ECACC No: 85110503); human retinoblasts (PER.C6 (CruCell, Leiden, The Netherlands)); monkey kidney CV1 line transformed by SV40 (COS-7, ATCC CRL 1651); human embryonic kidney line (293 cells subcloned for growth in suspension culture, Graham et al., J. Gen Virol., 36:59 (1977)); baby hamster kidney cells (BHK, ATCC CCL 10); Chinese hamster ovary cells+/−DHFR (CHO, Urlaub and Chasin, Proc. Natl. Acad. Sci. USA, 77:4216 (1980)); mouse sertoli cells (TM4, Mather, Biol. Reprod., 23:243-251 (1980)); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y Acad. Sci., 383:44-68 (1982)); MRC 5 cells; FS4 cells; and a human hepatoma line (Hep G2). In some embodiments, cell lines that may be utilized for VLP production include insect (e.g., Sf-9, Sf-21, Tn-368, Hi5) or plant (e.g., Leguminosa, cereal, or tobacco) cells. It will be appreciated in some embodiments, particularly when glycosylation is important for protein function, mammalian cells are preferable for protein expression and/or VLP production (see, e.g., Roldao A et al., 2010 Expt Rev Vaccines 9:1149-76).

It will be appreciated that a cell strain may be chosen which modulates the expression of the inserted sequences, or modifies and processes the gene product in a specific way. Different cells have characteristic and specific mechanisms for post-translational processing and modification of proteins and gene products. Appropriate cell lines or host systems can be chosen to ensure the correct modification and processing of the foreign protein expressed. Generally, eukaryotic host cells (also referred to as packaging cells (e.g., 293T human embryo kidney cells)) which possess appropriate cellular machinery for proper processing of the primary transcript, glycosylation and phosphorylation of the gene product may be used in accordance with the present invention.

VLPs may be purified according to known techniques, such as centrifugation, gradients, sucrose-gradient ultracentrifugation, tangential flow filtration and chromatography (e.g., ion exchange (anion and cation), affinity and sizing column chromatography), or differential solubility, among others. Alternatively or additionally, cell supernatant may be used directly, with no purification step. Additional entities, such as additional antigens or adjuvants may be added to purified VLPs.

In some embodiments, in order to produce the VLPs of the present disclosure, cells are co-transfected with two expression vectors, a first vector encoding a Gag-pp65 fusion polypeptide and a second vector encoding a gB envelope glycoprotein. The co-transfected HCMV gB plasmid enables particles budding from the cell surface to incorporate the gB protein into the lipid bilayer. As a result, “bivalent” VLPs comprising a HCMV pp65 non-structural protein and a HCMV gB envelope glycoprotein are produced. Typically, these VLPs have a gB content of 1/40^{th} to 1/15^{th} of the content of pp65, and typically 1/10^{th} to 1/20^{th} of the content of pp65.

The present inventors have previously reported development of HCMV VLP vaccines comprising a gB surface antigen presented in its native conformation which stimulated production of neutralizing antibodies, and a pp65 tegument protein which induced helper T cells (TH lymphocytes) and cytotoxic T cells (CTL) (WO 2013/068847). In a study using peripheral blood mononuclear cells from healthy subjects, this VLP was shown was shown to stimulate a CD4+ and a CD8+ T cell immune response which was superior to the response generated by recombinant gB and pp65 antigens alone (see Example 3).

The compositions of the present invention further comprise an adjuvant, granulocyte-macrophage colony-stimulating factor (GM-CSF). GM-CSF is a monomeric glycoprotein secreted by macrophages, T cells, mast cells, natural killer cells, endothelial cells and fibroblasts that functions as a cytokine. Studies have demonstrated that vaccination with irradiated tumor cells genetically modified to produce GM-CSF promoted potent anti-tumor immunity (Dranoff, G. Proc. Natl. Acad. Sci. 1993; 90: 3539-3542). GM-CSF has been shown to promote the development and maturation of antigen presenting cells and to skew the immune system toward Th1-type responses (Arellano, M. & Lonial, S. Biologics. 2008; 2:13-27). As a consequence, GM-CSF has been proposed as an adjuvant in cancer immunotherapy (Clive, K. S. Expert Rev Vaccines 2010; 9:529-525) including for the treatment of GBM (Schijns, V. E. Vaccine 2015; 33: 2690-2696).

In ex vivo studies using cells from healthy HCMV-positive subjects, the inventors of the present disclosure have shown that the inclusion of GM-CSF in the composition of the present disclosure enhances T cell production of interferon-γ (IFN-γ) by gB/pp65Gag VLP stimulation. As discussed above, IFN-γ has been identified as an anti-tumor effector molecule and mice deficient for IFN-γ or IFN-γ signaling are more susceptible to tumor formation. Thus, secretion of IFN-γ by tumor-reactive T cells represents a desirable biomarker that may be associated with greater efficacy. As further described in Example 4, cells from healthy subjects shows an increase in IFN-γ in the presence of the composition of the invention. These data support the use of gB/pp65Gag eVLPs formulated with GM-CSF to induce T cell reactivation towards a Th1 response with sustained IFN-γ production.

In order to further evaluate the immunological effects of an exemplary composition of the present disclosure, it was tested in naïve, healthy mice. The T cell response to treatment was assessed by measuring the change in IFN-γ-secreting CD4+ T cells. In splenocytes from treated mice, the composition of the invention was able to stimulate an HCMV-specific Th1 response as indicated by an increase in IFN-γ-secreting CD4+ T cells after ex vivo reactivation with recombinant pp65. These data demonstrate that the exemplary composition of the invention can induce de novo HCMV-specific T cell responses in naïve healthy animals, which confirms the results obtained in the ex vivo studies using cells obtained from healthy HCMV-positive subjects. However, results from rodent studies cannot demonstrate the effectiveness of the compositions of the present disclosure to stimulate a T cell response in human GBM patients showing immunity against HCMV. In order to assess whether the compositions of the present disclosure is effective of counteract the effects of immune dysregulation in GBM subjects, it is necessary to test the compositions in human GBM patients.

A composition of the invention was tested in human GBM patients in a Phase I-II dose escalation study. A total of 18 subjects with recurrent GBM were divided into three groups of six subjects each. Each group was assigned one of the following three dosages of the composition of the invention:
Low dose—(0.4 μg pp65 content) formulated with GM-CSF (200 μg) in 0.2 mL volume
Intermediate dose—(2 μg pp65 content) formulated with GM-CSF (200 μg) in 0.2 mL volume
High dose—(10 μg pp65 content) formulated with GM-CSF (200 μg) in 0.2 mL volume
   The composition was administered in two equal intradermal injections, given at separate sites, every four weeks until disease progression was established.

The patients were tested for antibodies against HCMV gB antigen prior to the first injection. Greater than half the patients showed no antibodies to gB, which indicated significant dysregulation of immunity against HCMV among the patient population.

Results from the Phase I-II clinical study show that each dose of the tested composition was able to stimulate an immune response in some GBM patients. Of the six subjects who showed an immune response to the composition, three were in the highest dose group, two were in the lowest dose group and one was in the intermediate dose group. Accordingly, at each dose, some subjects demonstrated an immune response. However, the low dose and the intermediate dose of the composition did not stimulate a T cell response in patients who showed immune dysregulation prior to the first injection, evidenced by a lack of detectable antibodies against the HCMV gB protein. Surprisingly, the highest dose of the composition was able to stimulate a T cell response in three out of five patients with significant immune dysregulation against HCMV. Patients who responded to the vaccine in all the dose groups also showed a physiological response in the form of disease stabilization. In particular, the vaccine responders demonstrated stable disease for greater than 12 weeks and they showed a 6.25 month improvement in median overall survival compared to non-responders. Most surprisingly, two patients in the highest dose group experienced a 60% reduction in the size of their primary tumours. The results show that each dose of the composition has the potential to induce an immune response. However, patients given the highest dose of the composition responded differently to that of patients given the low and intermediate doses. Specifically, the highest dose was able to overcome the immune dysregulation observed in GBM patients. With recovered immunity against HCMV, the responsive GBM patients were able to harness the ability of their T cells to prevent proliferation of GBM tumour cells and, as a result, experience improved overall survival time. Accordingly, the present disclosure describes significant advancements in the immunological treatment of GBM, specifically a composition which is able to achieve stable disease and longer survival times in certain GBM patients and, as well, is able to reverse HCMV immune dysregulation using treatment at a dose of 10 μg pp65 content.

Accordingly, in some embodiments, the present disclosure provides a composition for treatment of GBM comprising pp65-gB VLPs formulated with GM-CSF as an adjuvant in a dose of at least 0.4 μg pp65 and 200 μg GM-CSF. In some embodiments, the present disclosure further provides a composition for treatment of GBM comprising pp65-gB VLPs formulated with GM-CSF as an adjuvant in a dose of at least 10 μg pp65 and 200 μg GM-CSF, which dose is effective to stimulate a T cell response in GBM patients showing dysregulation of immunity against HCMV.

The present invention also provides pharmaceutical compositions comprising the VLPs described herein and GM-CSF. In some embodiments, compositions of the present invention further comprise at least one additional pharmaceutically acceptable excipient, adjuvant and/or carrier. Such pharmaceutical compositions may optionally be administered in combination with one or more additional therapeutically active substances.

In some embodiments, pharmaceutical compositions provided herein may be provided in a sterile injectable form (e.g., a form that is suitable for intradermal injection). For example, in some embodiments, pharmaceutical compositions are provided in a liquid dosage form that is suitable for injection. In some embodiments, pharmaceutical compositions are provided as powders (e.g. lyophilized and/or sterilized), optionally under vacuum, which are reconstituted with an aqueous diluent (e.g., water, buffer, salt solution, etc.) prior to injection. In some embodiments, pharmaceutical compositions are diluted and/or reconstituted in water, sodium chloride solution, sodium acetate solution, benzyl alcohol solution, phosphate buffered saline, etc. In some embodiments, powder should be mixed gently with the aqueous diluent (e.g., not shaken).

In some embodiments, provided pharmaceutical compositions comprise one or more pharmaceutically acceptable excipients (e.g., preservative, inert diluent, dispersing agent, surface active agent and/or emulsifier, buffering agent, etc.). Suitable excipients include, for example, water, saline, dextrose, sucrose, trehalose, glycerol, ethanol, or similar, and combinations thereof. Remington's The Science and Practice of Pharmacy, 21st Edition, A. R. Gennaro, (Lippincott, Williams & Wilkins, Baltimore, MD, 2006) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention. In some embodiments, pharmaceutical compositions comprise one or more preservatives. In some embodiments, pharmaceutical compositions comprise no preservative.

In some embodiments, pharmaceutical compositions are provided in a form that can be refrigerated and/or frozen. In some embodiments, reconstituted solutions and/or liquid dosage forms may be stored for a certain period of time after reconstitution (e.g., 2 hours, 12 hours, 24 hours, 2 days, 5 days, 7 days, 10 days, 2 weeks, a month, two months, or longer). In some embodiments, storage of VLP formulations for longer than the specified time results in VLP degradation.

Formulations of the pharmaceutical compositions described herein may be prepared by any method known or hereafter developed in the art of pharmacology. In some embodiments, such preparatory methods include the step of bringing active ingredient into association with one or more excipients and/or one or more other accessory ingredients, and then, if necessary and/or desirable, packaging the product into a desired single- or multi-dose unit.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to a dose which would be administered to a subject and/or a convenient fraction of such a dose such as, for example, one-half or one-third of such a dose. In a preferred embodiment of the invention, a dose of the composition of the invention is delivered in two separate half doses at the same time.

Relative amounts of active ingredient, pharmaceutically acceptable excipient, and/or any additional ingredients in a pharmaceutical composition in accordance with the invention may vary, depending upon the identity, size, and/or condition of the subject and/or depending upon the route by which the composition is to be administered.

In some embodiments, treatment includes multiple administrations, appropriately spaced in time, of the composition of the present disclosure. Compositions described herein will generally be administered for such a time as they continue to induce an immune response, or until such time as the patient experiences progression of their disease. In a preferred embodiment of the invention, the composition of the invention is administered every four weeks.

In some embodiments, the exact amount of an immunogenic composition to be administered is at least about 0.4 μg pp65 and about 200 μg GM-CSF and, for subjects showing immune dysregulation against HCMV, at least about 10 μg pp65 and about 200 μg GM-CSF. In some embodiments, an administered immunogenic composition comprises (i) at least about 0.4 μg pp65 (e.g., about 0.4 μg, about 0.5 μg, about 0.6 μg, about 0.7 μg, about 0.8 μg, about 0.9 μg, about 1 μg, about 2 μg or more, pp65), and (ii) at least about 200 μg GM-CSF (e.g., about 200 g, about 250 μg, about 300 μg, about 350 μg, about 400 μg, about 450 μg, about 500 μg, or more, GM-CSF). In some embodiments, for subjects showing immune dysregulation against HCMV, an administered immunogenic composition comprises (i) at least about 10 μg pp65 (e.g., about 10 μg, about 15 μg, about 20 μg, about 25 μg, about 30 μg, about 35 μg, about 40 μg, about 50 μg, or more, pp65), and (ii) at least about 200 μg GM-CSF (e.g., about 200 μg, about 250 μg, about 300 μg, about 350 μg, about 400 μg, about 450 μg, about 500 μg, or more, GM-CSF). The preferred dosage may vary from subject to subject and may depend on several factors. Thus, it will be appreciated that, in general, the precise dose used will be as determined by the prescribing physician and will depend not only on the weight of the subject, but also on the age of the subject and, possibly, the progression of the disease and the degree of immune dysregulation against HCMV in the patient.

In certain embodiments, provided compositions may be formulated for delivery parenterally, e.g., by injection. In such embodiments, administration may be, for example, intravenous, intramuscular, intradermal, or subcutaneous, or via by infusion or needleless injection techniques. In a preferred embodiment, the compositions are formulated for intradermal injection.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following examples. The examples are provided for illustrative purposes only. They are not to be construed as limiting the scope or content of the disclosure in any way.

### EXAMPLES

### Example 1: Construction of DNA Expression Plasmids

This Example describes development of expression plasmids and constructs for expression of recombinant HCMV gene sequences (gB and Gag/pp65 fusion gene sequences). A standard expression plasmid generally consists of a promoter sequence of mammalian origin, an intron sequence, a PolyAdenylation signal sequence (PolyA), a pUC origin of replication sequence (pUC—pBR322 is a colE1 origin/site of replication initiation and is used to replicate plasmid in bacteria such as E. Coli (DH5α)), and an antibiotic resistance gene as a selectable marker for plasmid plaque selection. Within the plasmid following the intron are a variety of restriction enzyme sites that can be used to splice in a gene or partial gene sequence of interest.

The Propol II expression plasmid contains the pHCMV (early promoter for HCMV), a Beta-Globin Intron (BGL Intron), a rabbit Globin polyAdenylation signal sequence (PolyA), a pUC origin of replication sequence (pUC—pBR322 is a colE1 origin/site of replication initiation and is used to replicate plasmid in bacteria such as E. coli (DH5α)), and an ampicillin resistance gene β-lactamase (Amp R—selectable marker for plasmid confers resistance to ampicillin (100 μg/ml).

To develop a Gag MMLV expression construct (“MLV-Gag”), a complementary DNA (cDNA) sequence encoding a Gag polyprotein of MMLV (Gag without its C terminus Pol sequence) (Seq ID NO: 3) was cloned in a Propol II expression vector. To develop a gB expression construct (“gB”), the full-length sequence of gB was codon-optimized for human expression (GenScript) and was cloned in a Propol II expression vector including the extracellular portion, transmembrane domain (TM) and cytoplasmic portion (Cyto) of gB. To develop a Gag/pp65 expression construct (“Gag/pp65”), a sequence encoding the Gag polyprotein of MMLV (Gag without its C terminus Pol sequence) was fused with the full-length sequence of pp65 codon-optimized for human expression (GenScript) and cloned in a Propol II expression vector.

DNA plasmids were amplified in competent E. coli (DH5α) and purified with endotoxin-free preparation kits according to standard protocols.

### Example 2: Production of Virus-Like Particles

This Example describes methods for production of virus-like particles containing various recombinant HCMV antigens described in Example 1.

293SF-3F6 cell line derived from HEK 293 cells are a proprietary suspension cell culture grown in serum-free chemically defined media (CA 2,252,972 and U.S. Pat. No. 6,210,922). The cells were transiently transfected using calcium phosphate methods with an MMLV-Gag/pp65 DNA expression plasmid and co-transfected with a gB DNA expression plasmid. Expression of HCMV antigens by the HEK 293 cells was confirmed by flow cytometry. After 48 to 72 hours of transfection, supernatants containing the VLPs were harvested and filtered through 0.45 m pore size membranes and further concentrated and purified by ultracentrifugation through a 20% sucrose cushion in a SW32 Beckman rotor (25,000 rpm, 2 hours, 4° C.). Pellets were resuspended in sterile endotoxin-free PBS (GIBCO) to obtain 500 times concentrated VLP stocks. Total protein was determined on an aliquot by a Bradford assay quantification kit (BioRad). Purified VLPs were stored at −80° C. until used. Each lot of purified VLPs was analyzed for the expression of gB, and MMLV-Gag/pp65 fusion protein by SDS-Page and Western Blot with specific antibodies to gB (CH 28 mouse monoclonal antibody to gB; Virusys Corporation; Pereira, L et al. 1984 Virology 139:73-86), and pp65 (CH12 mouse monoclonal antibody to UL83/pp65; Virusys Corporation; Pereira, L. et al. 1982 Infect Immun 36: 924-932). Antibodies were detected using enhanced chemilluminescence (ECL).

### Example 3: Stimulation of T Cells in PBMCs from Healthy HCMV-Positive Subjects Using gB/Pp65Gag VLPs

The objective of this study was to evaluate the ability of gB/pp65Gag VLPs produced as described in Example 2 and purified by sucrose cushion ultracentrifugation to activate pre-existing HCMV-specific CD4⁺ and CD8⁺ T cells in peripheral blood mononuclear cells (“PBMCs”) from healthy HCMV-positive subjects.

Human peripheral blood was obtained from CMV⁺ healthy donors. PBMCs were isolated from whole blood using Ficoll gradient separation and single use aliquots were created. PBMCs were used either fresh after separation or after storage at −170° C. Briefly, PBMCs were cultured at 1×10⁶ cells/mL in 4 mL PP culture tubes. gB/pp65 eVLPs and controls were added to the cells. Cells were cultured for 3 hours with stimulating agents prior to addition of Monensin and cultured for an additional 10 hours.

Potency was evaluated in ex vivo PBMC cultures in terms of the frequency of IFN-γ-secreting CD4⁺ and CD8⁺ T cells. Cells were collected and stained for surface antigens using PerCP-conjugated anti-CD3, PE-conjugated anti-CD4, and APC-conjugated anti-CD8 monoclonal antibodies. Cells were then permeabilized and fixed for intracellular staining with BV510-conjugated anti-IFNγ. Stained wells were analyzed by flow cytometry analysis on a FACS Accuri (Beckton-Dickinson). Using FlowJo software (TreeStar), gating was first performed on CD3⁺ cells to evaluate the proportion of IFN-γ secreting cells among either the CD3⁺CD4⁺ or the CD3⁺CD8⁺ populations.

Data are shown in Table 1 below. The data are shown as mean percentage of cells, after subtraction of background responses to stimulation with empty VLPs (in the case of compositions comprising gB/pp65 VLPs) or unstimulated cells (in the case of recombinant proteins).

TABLE 1

| | Mean % | Mean % |
|---|---|---|
| | IFNγ + T cells | IFNγ + T cells |
| | within CD4 + | within CD8 + |
| Composition | population | population |
| gB/pp65Gag VLP (2 μg/ml) | 0.4811 | 0.8422 |
| gB/pp65Gag VLP (1 mg/ml) | 0.4511 | 1.537 |
| Recomb gB (2 μg/ml) + pp65 | 0.0656 | 0.4789 |
| Recomb gB (1 μg/ml) + pp65 | 0.0889 | 0.8433 |

As shown above in Table 1, the bivalent gB/pp65Gag VLPs stimulate both CD4⁺ and CD8⁺ IFN-γ-secreting T cell responses ex vivo. A combination of recombinant gB and pp65 proteins was less effective than the bivalent VLPs at stimulating CD8⁺ and particularly CD4⁺ T cell responses in the PBMCs from healthy subjects.

### Example 4: Stimulation of T Cells in PBMCs from Healthy Subjects Using gB/Pp65Gag VLPs with GM-CSF

The objective of this study was to evaluate the ability of gB/pp65Gag VLPs formulated with GM-CSF to reactivate HCMV-specific ex vivo cultured T cells from healthy donors. T cell reactivation was evaluated either in terms of the frequency of IFN-γ-secreting CD4⁺ and CD8⁺ T cells or based on secretion of a panel of cytokines and chemokines.

PBMCs were isolated from 4 healthy donors using the method in Example 3 and were cultured with 2 doses of gB/pp65Gag VLPs and 2 doses of GM-CSF and stimulation controls. After culture, cells were collected for surface and intracellular staining as described in Example 3 or supernatants were collected for analysis of cytokines and chemokines using commercially available ELISA kits in accordance with manufacturers' instructions.

The results are shown in Table 2 as frequency of IFN-γ secreting T cells.

TABLE 2

| Composition | | |
|---|---|---|
| (each with 10 ng/ml GM-CSF) | CD4 + IFNγ | CD8 + IFNγ |
| gB/pp65Gag VLP (0.25 μg/ml gB) | 1.995 | 1.195 |
| gB/pp65Gag VLP (1 μg/ml) and | 1.643 | 1.133 |
| Empty GAG equal to 0.25 μg/ml gB | 0.308 | 0.120 |
| Empty GAG equal to 1 μg/ml gB | 0.300 | 0.143 |

As shown in Table 2, gB/pp65Gag VLPs formulated with GM-CSF stimulate IFN γ-producing CD4+ and CD8+ T cells in cultured PBMCs.

### Example 5: Characterization of Immune Response in Mice Using gB/Pp65Gag VLPs Formulated with GM-CSF

The objective of this study was to characterize the immune response induced in vivo in mice by bivalent gB/pp65Gag VLPs formulated with GM-CSF.

Twenty-four female Balb/C mice 6-8 weeks old were purchased from Charles River Laboratories (St-Constant, Quebec, Canada). Animals were allowed to acclimatize. The body weight of mice upon arrival was 18.1±0.42 g. Upon arrival, mice were randomized into 3 groups with 4 animals per group. The VLP dose in all groups was 0.5 μg gB based on gB content using ELISA and 2.5 μg/dose of murine GM-CSF. Mice were immunized at Days 0 and Day 28 with either a bivalent gB/pp65Gag VLP formulated with 5 μg/ml GM-CSF or with an empty VLP-GM-CSF control.

Collection of splenocytes and blood from 4 animals per group was scheduled at Day 10 after the second immunization. Freshly isolated cells were cultured in complete DMEM and stimulated for 16 hours with gB/pp65Gag VLP or recombinant gB, recombinant pp65 or empty Gag VLPs prior to flow cytometry analysis. The levels of expression of IFNγ in CD3+CD4+ T cells were evaluated using commercial kits.

The results are shown in Table 3 below.

TABLE 3

| | Mean % IFNγ + | Mean |
|---|---|---|
| | T cells | Anti-HCMV gB |
| | within CD4 + | Specific Total IgG |
| Composition | population | antibody titre |
| gB/pp65Gag VLP | 1.4 | 3691 |
| (1 μg/ml) plus | | |
| 5 μg/ml GM-CSF | | |
| 5 μg/ml GM-CSF | 0.25 | 38.38 |

As shown in Table 3, gB/pp65Gag VLPs can induce a CMV-specific Th1 response as indicated by the increase of IFN-γ-secreting CD4+ T cells after ex vivo reactivation with recombinant pp65. These data demonstrate that gB/pp65Gag eVLPs formulated with GM-CSF can induce de novo CMV-specific T cell responses in naïve animals, which confirm results obtained in ex vivo stimulation studies of PBMCs obtained from healthy subjects and GBM patients.

### Example 6: Clinical Trial of gB/Pp65Gag VLPs in Human GBM Patients

The gB/pp65Gag VLPs were tested in a dose-escalation study to define the safety, tolerability, and optimal dose level of an immunogenic composition comprising gB/pp65Gag VLPs formulated with GM-CSF as an adjuvant.

Eighteen adult subjects (18-70 years of age) with recurrent WHO grade IV GBM and unequivocal evidence of tumor recurrence (any number of recurrences) or progression after initial treatment that included surgery and radiation therapy, with or without temozolomide, were enrolled in the study. The subjects were divided into three groups of six participants each. Each group was administered one of the following three doses of the investigational product every 4 weeks until confirmed clinical disease progression:
Group 1: Low dose (0.4 μg gB/pp65Gag VLP) vaccine formulated with GM-CSF (200 μg) in 0.2 mL volume.
Group 2: Intermediate dose (2 μg gB/pp65Gag VLP) vaccine formulated with GM-CSF (200 μg) in 0.2 mL volume.
Group 3: High dose (10 μg gB/pp65Gag VLP) vaccine formulated with GM-CSF (200 μg) in 0.2 mL volume.

The investigational drug was administered in two equal intradermal injections at separate injection sites. When a subject met the criteria for clinical disease progression, the subject was withdrawn from study treatment and was no longer assessed for vaccine response. Clinical disease progression was monitored by measurement of tumour size using MRI. The subjects were monitored from the date of first injection until they showed documented tumour progression.

Response to the investigational drug was determined as follows:
Antibody titers against HCMV gB antigen by ELISA assay at baseline and 2 weeks after each dose of drug with results provided as serum IgG anti-gB antibody titers in baseline and post injection samples.
cellular immunity against HCMV gB and pp65 antigens using IFN-γ and IL-5 ELISPOT assessed at baseline and 2 weeks after each treatment. Results are provided as frequencies of IFN-γ and IL-5 spots/3×105 PBMCs post HCMV stimulation at baseline and after each treatment.
Progression free survival (PFS) from date of first dose to date of progression (or death.

Samples could not be obtained from three clinical trial subjects due to very rapid disease progression therefore no data was obtained for these subjects. For the remaining 15 subjects, the data are shown in Tables 4a and b (Low Dose), 5a and b (Intermediate Dose) and 6a and b (High Dose) below. Cellular immunity (CMI) data is expressed as spot forming cells (SFC) per 10⁶ PBMCs. Tumour response is shown for each month that the subject remained on the study as SD (stable disease), PD (progressed disease) or ? (test inconclusive).

TABLE 4a
Subjects Receiving Low Dose

| | | | | Peak # |
|---|---|---|---|---|
| | | Peak | | of gB specific |
| | | Antibody | gB CMI | T cells Secreting |
| | Baseline | Titre after | SFC/10⁶ | IFN-γ after |
| Subject | gB titer | Treatment | PBMCs | Treatment |
| 01-003 | EPT: 86,885 | 751,226 | SFC: 0 | 456 |
| 01-005 | EPT: 500 | 500 | SFC: 0 | 0 |
| 01-004 | EPT: 195412 | 207653 | SFC: 6 | 39 |
| 01-006 | EPT: 49,273 | 185,752 | SFC: 166 | 38 |
| 01-007 | EPT: 83,920 | 125,893 | SFC: 1 | 380 |
| 01-009 | EPT: 500 | 500 | SFC: 4 | 0 |

TABLE 4b
Subjects Receiving Low Dose

| | | Peak# | | |
|---|---|---|---|---|
| | | of pp65 | | |
| | | specific | | |
| | | T cells | | |
| | pp65 | Secreting | | Tumour |
| | CMI | IFN-γ | Vaccine | Response |
| | SFC/10⁶ | after | Induced | (shown |
| Subject | PBMCs | Treatment | Response | monthly) |
| 01-003 | SFC: 0 | 2,500 | Yes | SD→SD→SD→SD→SD |
| 01-005 | SFC: 0 | 0 | No | ?→?→PD |
| 01-004 | SFC: 0 | 0 | No | PD |
| 01-006 | SFC: 0 | 0 | No | PD |
| 01-007 | SFC: 0 | 847 | Yes | PD |
| 01-009 | SFC: 4 | 8 | No | PD |

TABLE 5a
Subjects Receiving Intermediate Dose

| | | | | Peak # of |
|---|---|---|---|---|
| | | Peak | | gB specific |
| | | Antibody | gB CMI | T cells |
| | Baseline | Titre after | SFC/10⁶ | Secreting IFN-γ |
| Subject | gB titer | Treatment | PBMCs | after Treatment |
| 01-012 | EPT: 124,595 | 494,373 | SFC: 1 | 55 |
| 01-013 | EPT: 500 | 500 | SFC: 0 | 5 |
| 01-016 | EPT: 500 | 500 | SFC: 8 | 0 |
| 03-001 | EPT: 500 | 500 | SFC: 5 | 21 |

TABLE 5b
Subjects Receiving Intermediate Dose

| | | Peak of | | |
|---|---|---|---|---|
| | | pp65 specific | | |
| | pp65 | T cells | | Tumour |
| | CMI | Secreting | Vaccine | Response |
| | SFC/10⁶ | IFN-γ | Induced | (shown |
| Subject | PBMCs | after Treatment | Response | monthly) |
| 01-012 | SFC: 181 | 2756 | Yes | SD→PD |
| 01-013 | SFC: 0 | 4 | No | PD |
| 01-016 | SFC: 66 | 0 | No | SD → PD |
| 03-001 | SFC: 1 | 55 | No | SD →voluntarily |
| | | | | withdrew after |
| | | | | first month |

TABLE 6a
Subjects Receiving High Dose

| | | | | Peak # of |
|---|---|---|---|---|
| | | Peak | | gB specific |
| | | Antibody | gB CMI | T cells |
| | Baseline | Titre after | SFC/10⁶ | Secreting IFN-γ |
| Subject | gB titer | Treatment | PBMCs | after Treatment |
| 01-017 | EPT: 500 | 500 | SFC: 0 | 0 |
| 03-003 | EPT: 500 | 10,977 | SFC: 5 | 0 |
| 01-018 | EPT: 500 | 500 | SFC: 0 | N/A |
| 03-004 | EPT: 500 | 4,349 | SFC: 0 | 70 |
| 03-006 | EPT: 500 | 500 | SFC: 33 | 75 |

TABLE 6b
Subjects Receiving High Dose

| | | Peak of pp65 | | |
|---|---|---|---|---|
| | pp65 | specific T cells | | Tumour |
| | CMI | Secreting | Vaccine | Response |
| | SFC/10⁶ | IFN-γ | Induced | (shown |
| Subject | PBMCs | after Treatment | Response | monthly) |
| 01-017 | SFC: 0 | 0 | No | PD |
| 03-003 | SFC: 24 | 159 | Yes | SD→ ? →SD |
| 01-018 | SFC: 0 | N/A | No | PD |
| 03-004 | SFC: 0 | 35 | Yes | SD→SD |
| 03-006 | SFC: 0 | 0 | No | SD →SD →SD→SD |

As can be seen from Tables 4 and 5, each of the low and intermediate stimulated a T cell response in GBM patients who had antibodies to the gB antigen prior to the first injection (i.e. at baseline). However, both the low and the intermediate dose failed to stimulate a T cell response in the patients who had no antibodies to gB prior to the first injection, evidence of dysregulation of HCMV immunity.

However, surprisingly, the high dose of the composition of the present disclosure stimulated an immune response in a majority (3 out of 5) of the patients with no antibodies to gB prior to the first injection (see Table 6). These patients had significant dysregulation of HCMV immunity prior to treatment. However, at the high dose, immune dysregulation was overcome and the patients mounted an antibody and a T cell response to HCMV antigens. Even more significantly, the patients that overcame HCMV-specific immune dyregulation after vaccination also showed a positive clinical response in terms of stabilization of tumor growth and disease progression.

The tumours of the patients with stabilized disease were measured using MRI. The results are shown in Table 7 below. Time is shown in weeks where time zero is the date of first treatment.

TABLE 7

| | Size (mm²) | Size (mm²) | Size (mm²) | Size (mm²) |
|---|---|---|---|---|
| Subject | Time = 0 | Time = 5-7 | Time = 11-13 | Time = 19 |
| 03-003 | 955 | 998 | 1828 | 2000 |
| 03-004 | 237 | 235 | 151 | 142 |
| | | | | (new lesion-385) |
| 03-006 | 186 | 128 | 102 | 77 |
| | | | new lesion-120 | 106 |

As can be seen in Table 7 above, two of the subjects with stable disease showed a decrease in the size of their primary tumours.

Clinical trial subjects were followed after the study for survival time until death. Table 8, below, shows the PFS and the overall survival time (in weeks) for each of the subjects who participated in the study, along with whether they responded to the study vaccine or not.

TABLE 8

| | PFS | Overall Survival Time | Vaccine Response |
|---|---|---|---|
| Subject | (weeks) | (weeks) | (yes or no) |
| 03-001 | 10 | 11.0 | No |
| 01-017 | 7 | 16 | No |
| 01-009 | 6 | 18 | No |
| 01-004 | 5 | 28 | No |
| 01-013 | 12 | 31 | No |
| 01-016 | 8 | 43 | No |
| 01-018 | 5 | 57 | No |
| 01-005 | 15 | 93 | No |
| 01-006 | 6 | 97 | No |
| 01-003 | 36 | 37 | Yes |
| 03-004 | 16 | 53 | Yes |
| 03-006 | 28 | 56 | Yes |
| 01-007 | 8 | 56 | Yes |
| 03-003 | 18 | 59 | Yes |
| 01-012 | 13 | 65 | Yes |

As can be seen in Table 8, overall survival rates for vaccine responders significantly exceeded the rates for non-responders, with a 25% overall survival rate at 12 months for vaccine non-responders vs. 83% overall survival rate at 12 months for vaccine responders. Median overall survival for vaccine non-responders was 31 weeks vs. 56 weeks for vaccine responders, an improvement of 6.25 months. Accordingly, response to the vaccine was highly correlated to improved survival time.

### EQUIVALENTS

It is to be understood that while the disclosure has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An immunotherapeutic composition for treatment of glioblastoma multiforme (GBM) in a human subject, said composition comprising
(i) a virus like particle (VLP) comprising:
(a) a fusion protein comprising an N-terminal portion of a gag protein found in a murine leukemia virus (MLV) fused upstream of a pp65 protein found in HCMV; and
(b) a polypeptide comprising a glycoprotein (gB) protein found in HCMV; and
(ii) GM-CSF;
wherein the VLP is present in an amount of at least 4 μg gB/pp65 Gag per dose.

2. The immunotherapeutic composition of claim 1 wherein the fusion protein comprises an amino acid sequence at least 85% identical to SEQ ID NO:4 and the polypeptide comprises an amino acid sequence that is at least 85% identical to SEQ ID NO:7.

3. The immunotherapeutic composition of claim 1 wherein the fusion protein comprises the amino acid sequence of SEQ ID NO:4 and the polypeptide comprises the amino acid sequence of SEQ ID NO:7.

4. The immunotherapeutic composition of claim 1 wherein the GM-CSF is present in an amount of at least 200 μg per dose.

5. A method of treating a subject having GBM, comprising administering to the subject the composition of claim 1.

6. An immunotherapeutic composition for treatment of glioblastoma multiforme (GBM) in a human subject, said composition comprising:
(i) a virus like particle (VLP) comprising:
(a) a fusion protein comprising an N-terminal portion of a gag protein found in a murine leukemia virus (MLV) fused upstream of a pp65 protein found in HCMV; and
(b) a polypeptide comprising a glycoprotein (gB) protein found in HCMV; and
(ii) GM-CSF;
wherein the VLP is present in an amount of at least 10 μg gB/pp65 Gag per dose.

7. The immunotherapeutic composition of claim 6, wherein the fusion protein comprises an amino acid sequence at least 85% identical to SEQ ID NO:4 and the polypeptide comprises an amino acid sequence that is at least 85% identical to SEQ ID NO:7.

8. The immunotherapeutic composition of claim 7, wherein the fusion protein comprises the amino acid sequence of SEQ ID NO:4 and the polypeptide comprises the amino acid sequence of SEQ ID NO:7.

9. The immunotherapeutic composition of claim 6, wherein the GM-CSF is present in an amount of at least 200 μg per dose.

10. A method of treating a subject having GBM, comprising administering to the subject the composition of claim 6.

11. The method of claim 10, wherein the subject has dysregulation of immunity to HCMV, said dysregulation measured by a lack of detectable antibody response to HCMV gB protein.


SEQUENCE LISTING (10 sequences)

SEQ ID NO: 1 - protein, 538 aa, Murine leukemia virus
MGQTVTTPLSLTLGHWKDVERIAHNQSVDVKKRRWVTFCSAEWPTFNVGWPRDGTFNRDLITQVKIKVFSPGPHGHPDQVPYIVTWEALAFDPPPWVKPFVHPKPPPPLPPSAPSLPLEPPRSTPPRSSLYPALTPSLGAKPKPQVLSDSGGPLIDLLTEDPPPYRDPRPPPSDRDGNGGEATPAGEAPDPSPMASRLRGRREPPVADSTTSQAFPLRAGGNGQLQYWPFSSSDLYNWKNNNPSFSEDPGKLTALIESVLITHQPTWDDCQQLLGTLLTGEEKQRVLLEARKAVRGDDGRPTQLPNEVDAAFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLERLEDLKNKTLGDLVREAEKIFNKRETPEEREERIRRETEEKEERRRTEDEQKEKERDRRRHREMSKLLATVVSGQKQDRQGGERRRSQLDRDQCAYCKEKGHWAKDCPKKPRGPRGPRPQTSLLTLDD

SEQ ID NO: 2 - DNA, 1614 bp, Murine leukemia virus
  source 1..1614: mol_type = unassigned DNA
atgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgac

SEQ ID NO: 3 - DNA, 1614 bp, synthetic construct
  misc_feature 1..1614: note = Description of Artificial Sequence: Synthetic polynucleotide
  source 1..1614: mol_type = other DNA
atgggacagaccgtcacaacacccctgagcctgaccctgggacattggaaagacgtggagaggatcgcacataaccagagcgtggacgtgaagaaacggagatgggtcacattctgcagtgctgagtggccaacttttaatgtgggatggccccgagacggcactttcaacagggatctgatcacccaggtgaagatcaaggtctttagcccaggacctcacggacatccagaccaggtgccttatatcgtcacctgggaggcactggccttcgatccccctccatgggtgaagccatttgtccacccaaaaccacctccaccactgcctccaagtgccccttcactgccactggaaccaccccggagcacaccaccccgcagctccctgtatcctgctctgactccatctctgggcgcaaagccaaaaccacaggtgctgagcgactccggaggaccactgattgacctgctgacagaggaccccccaccataccgagatcctcggcctccaccaagcgaccgcgatggaaatggaggagaggctactcctgccggcgaagcccctgacccatctccaatggctagtaggctgcgcggcaggcgcgagcctccagtggcagatagcaccacatcccaggccttccctctgagggctgggggaaatgggcagctccagtattggccattttctagttcagacctgtacaactggaagaacaataacccctctttcagtgaggaccccggcaaactgaccgccctgatcgaatccgtgctgattacccatcagcccacatgggacgattgtcagcagctcctgggcaccctgctgaccggagaggaaaagcagcgcgtgctgctggaggctcgcaaagcagtccgaggggacgatggacggcccacacagctccctaatgaggtggacgccgcttttccactggaaagacccgactgggattatactacccaggcagggagaaaccacctggtccattacaggcagctcctgctggcaggcctgcagaatgccgggagatcccccaccaacctggccaaggtgaaaggcatcacacaggggcctaatgagtcaccaagcgcctttctggagaggctgaaggaagcttaccgacggtataccccatacgaccctgaggaccccggacaggaaacaaacgtctccatgtctttcatctggcagtctgccccagacattgggcggaagctggagagactggaagacctgaagaacaagaccctgggcgacctggtgcgggaggctgaaaagatcttcaacaaacgggagacccccgaggaaagagaggaaaggattagaagggaaactgaggaaaaggaggaacgccgacggaccgaggacgaacagaaggagaaagaacgagatcggcggcggcaccgggagatgtcaaagctgctggccaccgtggtcagcggacagaaacaggacagacagggaggagagcgacggagaagccagctcgacagggatcagtgcgcatactgtaaggaaaaaggccattgggccaaggattgccccaaaaagccaagaggaccaagaggaccaagaccacagacatcactgctgaccctggacgac

SEQ ID NO: 4 - protein, 1099 aa, synthetic construct
  REGION 1..1099: note = Description of Artificial Sequence: Synthetic polypeptide
MGQTVTTPLSLTLGHWKDVERIAHNQSVDVKKRRWVTFCSAEWPTFNVGWPRDGTFNRDLITQVKIKVFSPGPHGHPDQVPYIVTWEALAFDPPPWVKPFVHPKPPPPLPPSAPSLPLEPPRSTPPRSSLYPALTPSLGAKPKPQVLSDSGGPLIDLLTEDPPPYRDPRPPPSDRDGNGGEATPAGEAPDPSPMASRLRGRREPPVADSTTSQAFPLRAGGNGQLQYWPFSSSDLYNWKNNNPSFSEDPGKLTALIESVLITHQPTWDDCQQLLGTLLTGEEKQRVLLEARKAVRGDDGRPTQLPNEVDAAFPLERPDWDYTTQAGRNHLVHYRQLLLAGLQNAGRSPTNLAKVKGITQGPNESPSAFLERLKEAYRRYTPYDPEDPGQETNVSMSFIWQSAPDIGRKLERLEDLKNKTLGDLVREAEKIFNKRETPEEREERIRRETEEKEERRRTEDEQKEKERDRRRHREMSKLLATVVSGQKQDRQGGERRRSQLDRDQCAYCKEKGHWAKDCPKKPRGPRGPRPQTSLLTLDDCESRGRRCPEMISVLGPISGHVLKAVFSRGDTPVLPHETRLLQTGIHVRVSQPSLILVSQYTPDSTPCHRGDNQLQVQHTYFTGSEVENVSVNVHNPTGRSICPSQEPMSIYVYALPLKMLNIPSINVHHYPSAAERKHRHLPVADAVIHASGKQMWQARLTVSGLAWTRQQNQWKEPDVYYTSAFVFPTKDVALRHVVCAHELVCSMENTRATKMQVIGDQYVKVYLESFCEDVPSGKLFMHVTLGSDVEEDLTMTRNPQPFMRPHERNGFTVLCPKNMIIKPGKISHIMLDVAFTSHEHFGLLCPKSIPGLSISGNLLMNGQQIFLEVQAIRETVELRQYDPVAALFFFDIDLLLQRGPQYSEHPTFTSQYRIQGKLEYRHTWDRHDEGAAQGDDDVWTSGSDSDEELVTTERKTPRVTGGGAMAGASTSAGRKRKSASSATACTAGVMTRGRLKAESTVAPEEDTDEDSDNEIHNPAVFTWPPWQAGILARNLVPMVATVQGQNLKYQEFFWDANDIYRIFAELEGVWQPAAQPKRRRHRQDALPGPCIASTPKKHRG

SEQ ID NO: 5 - DNA, 3300 bp, synthetic construct
  misc_feature 1..3300: note = Description of Artificial Sequence: Synthetic polynucleotide
  source 1..3300: mol_type = other DNA
atgggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccaaacctaaacctcaagttctttctgacagtggggggccgctcatcgacctacttacagaagaccccccgccttatagggacccaagaccacccccttccgacagggacggaaatggtggagaagcgacccctgcgggagaggcaccggacccctccccaatggcatctcgcctacgtgggagacgggagccccctgtggccgactccactacctcgcaggcattccccctccgcgcaggaggaaacggacagcttcaatactggccgttctcctcttctgacctttacaactggaaaaataataacccttctttttctgaagatccaggtaaactgacagctctgatcgagtctgttctcatcacccatcagcccacctgggacgactgtcagcagctgttggggactctgctgaccggagaagaaaaacaacgggtgctcttagaggctagaaaggcggtgcggggcgatgatgggcgccccactcaactgcccaatgaagtcgatgccgcttttcccctcgagcgcccagactgggattacaccacccaggcaggtaggaaccacctagtccactatcgccagttgctcctagcgggtctccaaaacgcgggcagaagccccaccaatttggccaaggtaaaaggaataacacaagggcccaatgagtctccctcggccttcctagagagacttaaggaagcctatcgcaggtacactccttatgaccctgaggacccagggcaagaaactaatgtgtctatgtctttcatttggcagtctgccccagacattgggagaaagttagagaggttagaagatttaaaaaacaagacgcttggagatttggttagagaggcagaaaagatctttaataaacgagaaaccccggaagaaagagaggaacgtatcaggagagaaacagaggaaaaagaagaacgccgtaggacagaggatgagcagaaagagaaagaaagagatcgtaggagacatagagagatgagcaagctattggccactgtcgttagtggacagaaacaggatagacagggaggagaacgaaggaggtcccaactcgatcgcgaccagtgtgcctactgcaaagaaaaggggcactgggctaaagattgtcccaagaaaccacgaggacctcggggaccaagaccccagacctccctcctgaccctagatgactgtgagtcgcgcggtcgccgttgtcccgaaatgatatccgtactgggtcccatttcggggcacgtgctgaaagccgtgtttagtcgcggcgacacgccggtgctgccgcacgagacgcgactcctgcagacgggtatccacgtgcgcgtgagccagccctcgctgatcctggtgtcgcagtacacgcccgactcgacgccatgccaccgcggcgacaatcagctgcaggtgcagcacacgtactttacgggcagcgaggtggagaacgtgtcggtcaacgtgcacaaccccacgggccggagcatctgccccagccaagagcccatgtcgatctatgtgtacgcgctgccgctcaagatgctgaacatccccagcatcaacgtgcaccactacccgtcggcggccgagcgcaaacaccgacacctgcccgtagctgacgctgtgattcacgcgtcgggcaagcagatgtggcaggcgcgtctcacggtctcgggactggcctggacgcgtcagcagaaccagtggaaagagcccgacgtctactacacgtcagcgttcgtgtttcccaccaaggacgtggcactgcggcacgtggtgtgcgcgcacgagctggtttgctccatggagaacacgcgcgcaaccaagatgcaggtgataggtgaccagtacgtcaaggtgtacctggagtccttctgcgaggacgtgccctccggcaagctctttatgcacgtcacgctgggctctgacgtggaagaggacctgacgatgacccgcaacccgcaacccttcatgcgcccccacgagcgcaacggctttacggtgttgtgtcccaaaaatatgataatcaaaccgggcaagatctcgcacatcatgctggatgtggcttttacctcacacgagcattttgggctgctgtgtcccaagagcatcccgggcctgagcatctcaggtaacctattgatgaacgggcagcagatcttcctggaggtgcaagcgatacgcgagaccgtggaactgcgtcagtacgatcccgtggctgcgctcttctttttcgatatcgacttgctgctgcagcgcgggcctcagtacagcgaacaccccaccttcaccagccagtatcgcatccagggcaagcttgagtaccgacacacctgggaccggcacgacgagggtgccgcccagggcgacgacgacgtctggaccagcggatcggactccgacgaggaactcgtaaccaccgagcgcaagacgccccgcgttaccggcggcggcgccatggcgggcgcctccacttccgcgggccgcaaacgcaaatcagcatcctcggcgacggcgtgcacggcgggcgttatgacacgcggccgccttaaggccgagtccaccgtcgcgcccgaagaggacaccgacgaggattccgacaacgaaatccacaatccggccgtgttcacctggccgccctggcaggccggcatcctggcccgcaacctggtgcccatggtggctacggttcagggtcagaatctgaagtaccaggagttcttctgggacgccaacgacatctaccgcatcttcgccgaattggaaggcgtatggcagcccgctgcgcaacccaaacgtcgccgccaccggcaagacgccttgcccgggccatgcatcgcctcgacgcccaaaaagcaccgaggttag

SEQ ID NO: 6 - DNA, 3300 bp, synthetic construct
  misc_feature 1..3300: note = Description of Artificial Sequence: Synthetic polynucleotide
  source 1..3300: mol_type = other DNA
atgggacagacagtcactacacccctgagcctgacactgggacattggaaagacgtggagaggattgcacataaccagagcgtggacgtgaagaaacggagatgggtcaccttttgctccgccgagtggccaacattcaatgtgggatggccccgagatggcaccttcaaccgggacctgatcactcaggtgaagatcaaggtcttctctccaggaccccacggccatccagatcaggtgccctacatcgtcacctgggaggctctggcatttgacccccctccatgggtgaagcctttcgtccacccaaaaccacctccaccactgcctccatctgcccctagtctgccactggaaccccctcggtcaaccccacccagaagctccctgtatcccgcactgacacctagcctgggggccaagcctaaaccacaggtgctgtctgatagtggcgggcctctgatcgatctgctgaccgaggaccctccaccataccgcgacccacgacctccaccaagcgaccgggacggaaacggaggagaggctacacccgcaggcgaagcccccgatcctagtccaatggcatcaaggctgcgcgggaggcgcgaacctccagtggccgactcaaccacaagccaggcatttccactgagggccgggggaaatggacagctccagtattggcccttctctagttcagatctgtacaactggaagaacaataaccctagcttcagcgaggacccaggcaaactgaccgccctgatcgaatccgtgctgattacccaccagcccacatgggacgattgtcagcagctcctgggcaccctgctgaccggagaggaaaagcagagagtgctgctggaggctaggaaagcagtccgcggggacgatggaaggccaacacagctccccaatgaggtggatgccgctttccctctggaacggccagattgggactatactacccaggctggacgcaaccacctggtgcattaccggcagctcctgctggctggactgcagaatgcagggcgcagccccactaacctggccaaggtgaaaggaatcacccagggccccaatgagtccccttctgcattcctggagcggctgaaggaagcctaccgacggtatactccctacgatcctgaggacccaggccaggaaaccaacgtgagtatgagcttcatctggcagtccgctcctgacattggccgaaaactggagcggctggaagatctgaagaacaagaccctgggcgacctggtgcgggaggcagaaaagatcttcaacaaaagggagactccagaggaacgggaggaaagaattagaagggaaacagaggaaaaggaggaacgccgacggactgaggatgaacagaaggagaaagaaagagaccggcggcggcaccgggagatgtctaagctgctggccaccgtggtcagtggccagaaacaggatcgacagggaggagagcgacggagaagccagctcgatcgggaccagtgcgcctattgtaaggaaaaagggcattgggctaaggactgccccaagaaacccagaggcccacgcgggccccgacctcagacttccctgctgaccctggacgattgcgagagccggggccggcggtgcccagaaatgatctctgtgctggggcccattagtggacatgtgctgaaggccgtcttctccaggggagacacccccgtgctgcctcacgagactcgactgctgcagaccggcatccatgtgcgggtctcccagccctctctgattctggtgtcacagtatacaccagatagcactccctgccacagaggagacaatcagctccaggtgcagcatacctactttacaggctccgaggtcgaaaacgtgtctgtcaatgtgcacaaccctaccggcaggagcatctgtcctagccaggagccaatgagcatctacgtgtacgccctgcctctgaagatgctgaatatcccatcaattaacgtccaccattaccctagcgcagccgaacggaagcacagacatctgccagtggccgacgctgtcatccatgccagcggcaaacagatgtggcaggcaagactgaccgtgtccgggctggcctggacaaggcagcagaatcagtggaaggagcccgacgtgtactataccagcgccttcgtgttccctaccaaagacgtggccctgagacatgtggtgtgcgcacatgagctggtgtgcagcatggaaaacactagggccaccaagatgcaggtcatcggcgatcagtatgtcaaagtgtacctggagagtttttgcgaagacgtgccatcagggaagctgttcatgcatgtgaccctgggcagcgatgtcgaggaagacctgaccatgacaagaaatccacagccctttatgagaccccacgagaggaatgggttcactgtgctgtgccccaagaacatgatcattaagcctggaaaaatcagtcatattatgctggatgtggcctttacatcacacgagcatttcggactgctgtgccccaaatccatccctggactgagcatttccggcaatctgctgatgaacggccagcagatcttcctggaagtgcaggccatccgggagaccgtcgaactgcgacagtatgacccagtggctgcactgttctttttcgacatcgacctgctgctgcagcgaggaccacagtacagcgagcaccctacttttacctcccagtatcggattcaggggaagctggagtacaggcacacctgggatcgccatgacgaaggagccgctcagggggacgatgacgtgtggacatctggcagtgattcagacgaggaactggtgacaactgagcgaaaaaccccccgggtgacaggaggaggggcaatggcaggggccagcaccagcgcagggcggaagcgaaaaagcgccagcagcgccacagcatgtaccgccggcgtgatgactagaggaaggctgaaggccgagtctacagtcgctcccgaggaagatactgacgaggatagtgacaatgaaatccacaaccccgccgtgttcacctggccaccttggcaggcagggattctggctcgcaacctggtccccatggtggcaaccgtccagggacagaatctgaagtatcaggagtttttctgggatgctaacgacatctaccggatttttgcagagctggaaggcgtgtggcagccagcagcccagcccaaacgacggagacatcgacaggacgctctgccaggaccttgtatcgccagcacaccaaagaagcacaggggctaa

SEQ ID NO: 7 - DNA, 3300 bp, synthetic construct
  misc_feature 1..3300: note = Description of Artificial Sequence: Synthetic polynucleotide
  source 1..3300: mol_type = other DNA
atgggacagaccgtcacaacacccctgagcctgaccctgggacattggaaagacgtggagaggatcgcacataaccagagcgtggacgtgaagaaacggagatgggtcacattctgcagtgctgagtggccaacttttaatgtgggatggccccgagacggcactttcaacagggatctgatcacccaggtgaagatcaaggtctttagcccaggacctcacggacatccagaccaggtgccttatatcgtcacctgggaggcactggccttcgatccccctccatgggtgaagccatttgtccacccaaaaccacctccaccactgcctccaagtgccccttcactgccactggaaccaccccggagcacaccaccccgcagctccctgtatcctgctctgactccatctctgggcgcaaagccaaaaccacaggtgctgagcgactccggaggaccactgattgacctgctgacagaggaccccccaccataccgagatcctcggcctccaccaagcgaccgcgatggaaatggaggagaggctactcctgccggcgaagcccctgacccatctccaatggctagtaggctgcgcggcaggcgcgagcctccagtggcagatagcaccacatcccaggccttccctctgagggctgggggaaatgggcagctccagtattggccattttctagttcagacctgtacaactggaagaacaataacccctctttcagtgaggaccccggcaaactgaccgccctgatcgaatccgtgctgattacccatcagcccacatgggacgattgtcagcagctcctgggcaccctgctgaccggagaggaaaagcagcgcgtgctgctggaggctcgcaaagcagtccgaggggacgatggacggcccacacagctccctaatgaggtggacgccgcttttccactggaaagacccgactgggattatactacccaggcagggagaaaccacctggtccattacaggcagctcctgctggcaggcctgcagaatgccgggagatcccccaccaacctggccaaggtgaaaggcatcacacaggggcctaatgagtcaccaagcgcctttctggagaggctgaaggaagcttaccgacggtataccccatacgaccctgaggaccccggacaggaaacaaacgtctccatgtctttcatctggcagtctgccccagacattgggcggaagctggagagactggaagacctgaagaacaagaccctgggcgacctggtgcgggaggctgaaaagatcttcaacaaacgggagacccccgaggaaagagaggaaaggattagaagggaaactgaggaaaaggaggaacgccgacggaccgaggacgaacagaaggagaaagaacgagatcggcggcggcaccgggagatgtcaaagctgctggccaccgtggtcagcggacagaaacaggacagacagggaggagagcgacggagaagccagctcgacagggatcagtgcgcatactgtaaggaaaaaggccattgggccaaggattgccccaaaaagccaagaggaccaagaggaccaagaccacagacatcactgctgaccctggacgactgcgagagccggggccggcggtgcccagaaatgatctctgtgctggggcccattagtggacatgtgctgaaggccgtcttctccaggggagacacccccgtgctgcctcacgagactcgactgctgcagaccggcatccatgtgcgggtctcccagccctctctgattctggtgtcacagtatacaccagatagcactccctgccacagaggagacaatcagctccaggtgcagcatacctactttacaggctccgaggtcgaaaacgtgtctgtcaatgtgcacaaccctaccggcaggagcatctgtcctagccaggagccaatgagcatctacgtgtacgccctgcctctgaagatgctgaatatcccatcaattaacgtccaccattaccctagcgcagccgaacggaagcacagacatctgccagtggccgacgctgtcatccatgccagcggcaaacagatgtggcaggcaagactgaccgtgtccgggctggcctggacaaggcagcagaatcagtggaaggagcccgacgtgtactataccagcgccttcgtgttccctaccaaagacgtggccctgagacatgtggtgtgcgcacatgagctggtgtgcagcatggaaaacactagggccaccaagatgcaggtcatcggcgatcagtatgtcaaagtgtacctggagagtttttgcgaagacgtgccatcagggaagctgttcatgcatgtgaccctgggcagcgatgtcgaggaagacctgaccatgacaagaaatccacagccctttatgagaccccacgagaggaatgggttcactgtgctgtgccccaagaacatgatcattaagcctggaaaaatcagtcatattatgctggatgtggcctttacatcacacgagcatttcggactgctgtgccccaaatccatccctggactgagcatttccggcaatctgctgatgaacggccagcagatcttcctggaagtgcaggccatccgggagaccgtcgaactgcgacagtatgacccagtggctgcactgttctttttcgacatcgacctgctgctgcagcgaggaccacagtacagcgagcaccctacttttacctcccagtatcggattcaggggaagctggagtacaggcacacctgggatcgccatgacgaaggagccgctcagggggacgatgacgtgtggacatctggcagtgattcagacgaggaactggtgacaactgagcgaaaaaccccccgggtgacaggaggaggggcaatggcaggggccagcaccagcgcagggcggaagcgaaaaagcgccagcagcgccacagcatgtaccgccggcgtgatgactagaggaaggctgaaggccgagtctacagtcgctcccgaggaagatactgacgaggatagtgacaatgaaatccacaaccccgccgtgttcacctggccaccttggcaggcagggattctggctcgcaacctggtccccatggtggcaaccgtccagggacagaatctgaagtatcaggagtttttctgggatgctaacgacatctaccggatttttgcagagctggaaggcgtgtggcagccagcagcccagcccaaacgacggagacatcgacaggacgctctgccaggaccttgtatcgccagcacaccaaagaagcacaggggctaa

SEQ ID NO: 8 - protein, 907 aa, Human cytomegalovirus
MESRIWCLVVCVNLCIVCLGAAVSSSSTRGTSATHSHHSSHTTSAAHSRSGSVSQRVTSSQTVSHGVNETIYNTTLKYGDVVGVNTTKYPYRVCSMAQGTDLIRFERNIVCTSMKPINEDLDEGIMVVYKRNIVAHTFKVRVYQKVLTFRRSYAYIHTTYLLGSNTEYVAPPMWEIHHINSHSQCYSSYSRVIAGTVFVAYHRDSYENKTMQLMPDDYSNTHSTRYVTVKDQWHSRGSTWLYRETCNLNCMVTITTARSKYPYHFFATSTGDVVDISPFYNGTNRNASYFGENADKFFIFPNYTIVSDFGRPNSALETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETTGGLVVFWQGIKQKSLVELERLANRSSLNLTHNRTKRSTDGNNATHLSNMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSINVFDLEEIMREFNSYKQRVKYVEDKVVDPLPPYLKGLDDLMSGLGAAGKAVGVAIGAVGGAVASVVEGVATFLKNPFGAFTIILVAIAVVIIIYLIYTRQRRLCMQPLQNLFPYLVSADGTTVTSGNTKDTSLQAPPSYEESVYNSGRKGPGPPSSDASTAAPPYTNEQAYQMLLALVRLDAEQRAQQNGTDSLDGQTGTQDKGQKPNLLDRLRHRKNGYRHLKDSDEEENV

SEQ ID NO: 9 - DNA, 2724 bp, Human cytomegalovirus
  source 1..2724: mol_type = unassigned DNA
atggagtcaaggatttggtgcctggtcgtgtgcgtcaatctgtgcatcgtctgtctgggggctgccgtgtcatcaagttctacaagaggcaccagcgccacccactcacaccatagctcccataccacatccgccgctcactcccggtctggcagcgtgagccagagagtcacatctagtcagaccgtgagccacggggtcaacgagaccatctacaatactaccctgaagtatggcgacgtggtcggggtgaacacaactaaatacccatatagggtctgcagtatggcccagggcactgatctgattagattcgaaaggaacatcgtgtgcaccagcatgaagcccattaatgaggacctggatgaagggatcatggtggtctacaaacgcaatattgtggcccataccttcaaggtgcgagtctatcagaaagtgctgacatttcggagatcttacgcatatatccacaccacatacctgctggggagtaacaccgagtatgtggctccccctatgtgggaaattcaccatatcaatagccattcccagtgctactcaagctacagcagagtgatcgctggaacagtgttcgtcgcataccacagagactcttatgagaacaagactatgcagctcatgcccgacgattacagcaatacacattccactagatatgtgacagtcaaagatcagtggcactcaaggggcagcacctggctgtaccgcgagacatgcaacctgaattgtatggtgactatcactaccgctagatccaagtacccctatcacttctttgcaacttccaccggggacgtggtcgatatttctcctttctacaacggcacaaaccggaatgcatcttattttggggagaacgccgacaagttctttattttcccaaattacaccatcgtgtctgattttggcagacccaacagtgccctggagacacatcgactggtggcattcctggaacgggccgactccgtcatttcttgggacatccaggatgagaagaatgtgacctgccagctcaccttctgggaggccagcgaacgcaccatccgatccgaggctgaagattcttaccacttctcctctgccaaaatgacagctacttttctgagcaagaaacaggaggtgaacatgtctgacagtgctctggattgcgtgcgggacgaagcaattaataagctgcagcagatcttcaacacatcatacaaccagacttacgagaagtacggaaacgtgagcgtcttcgaaacaactggcgggctggtggtcttttggcagggcatcaagcagaaatccctggtggagctggaaaggctggccaatcgcagttcactgaacctgactcataatcggaccaagagatctacagacggaaacaatgccacacatctgtctaacatggagagtgtgcacaatctggtctacgctcagctccagtttacctacgacacactgagaggctatattaacagggcactggcccagatcgctgaagcatggtgcgtggatcagaggcgcaccctggaggtcttcaaggaactgtccaaaatcaacccttcagcaattctgagcgccatctacaataagccaattgcagccaggtttatgggagacgtgctgggcctggccagttgcgtcactatcaaccagacctcagtgaaggtcctgcgcgatatgaatgtgaaagagagtcccggcagatgctattcacggcctgtggtcatcttcaactttgctaatagctcctacgtgcagtatggacagctcggcgaggacaacgaaattctgctggggaatcacaggaccgaggaatgtcagctccctagcctgaagattttcatcgctggaaactccgcatacgagtatgtggattacctgttcaagcggatgattgacctgtctagtatctccactgtggattctatgattgccctggacatcgatccactggaaaataccgacttcagggtgctggagctgtatagccagaaggaactgcgctccatcaacgtgttcgatctggaggaaattatgagagagtttaatagctacaagcagagggtgaaatatgtcgaagataaggtggtcgaccccctgccaccctacctgaaaggcctggacgatctgatgagcgggctgggagctgcagggaaggcagtgggagtcgctatcggcgcagtgggaggagccgtggccagcgtggtcgagggagtggcaacattcctgaaaaaccccttcggggccttcaccatcattctggtggcaatcgccgtggtcatcattatctacctgatctacacaaggcagcggcggctgtgcatgcagcctctgcagaacctgtttccatacctggtgagcgccgacgggaccacagtcacctcaggaaatactaaggatacctctctgcaggcccccccaagttacgaggaatcagtgtataacagcggcagaaaaggaccaggaccaccttcaagcgacgccagcactgccgctccaccctacaccaatgagcaggcctatcagatgctgctggctctggtgcgcctggatgccgaacagcgagctcagcagaacgggaccgactccctggatggacagaccggaacacaggacaagggacagaaacctaatctgctggatcggctgcggcacagaaaaaacgggtataggcacctgaaggactccgacgaagaagaaaatgtctaa

SEQ ID NO: 10 - DNA, 2724 bp, synthetic construct
  misc_feature 1..2724: note = Description of Artificial Sequence: Synthetic polynucleotide
  source 1..2724: mol_type = other DNA
atggaatccaggatctggtgcctggtagtctgcgttaacttgtgtatcgtctgtctgggtgctgcggtttcctcatcttctactcgtggaacttctgctactcacagtcaccattcctctcatacgacgtctgctgctcattctcgatccggttcagtctctcaacgcgtaacttcttcccaaacggtcagccatggtgttaacgagaccatctacaacactaccctcaagtacggagatgtggtgggggtcaacaccaccaagtacccctatcgcgtgtgttctatggcacagggtacggatcttattcgctttgaacgtaatatcgtctgcacctcgatgaagcccatcaatgaagacctggacgagggcatcatggtggtctacaaacgcaacatcgtcgcgcacacctttaaggtacgagtctaccagaaggttttgacgtttcgtcgtagctacgcttacatccacaccacttatctgctgggcagcaacacggaatacgtggcgcctcctatgtgggagattcatcatatcaacagtcacagtcagtgctacagttcctacagccgcgttatagcaggcacggttttcgtggcttatcatagggacagctatgaaaacaaaaccatgcaattaatgcccgacgattattccaacacccacagtacccgttacgtgacggtcaaggatcaatggcacagccgcggcagcacctggctctatcgtgagacctgtaatctgaattgtatggtgaccatcactactgcgcgctccaagtatccctatcattttttcgcaacttccacgggtgatgtggttgacatttctcctttctacaacggaactaatcgcaatgccagctattttggagaaaacgccgacaagtttttcatttttccgaactacactatcgtctccgactttggaagaccgaattctgcgttagagacccacaggttggtggcttttcttgaacgtgcggactcagtgatctcctgggatatacaggacgagaagaatgttacttgtcaactcactttctgggaagcctcggaacgcaccattcgttccgaagccgaggactcgtatcacttttcttctgccaaaatgaccgccactttcttatctaagaagcaagaggtgaacatgtccgactctgcgctggactgtgtacgtgatgaggccataaataagttacagcagattttcaatacttcatacaatcaaacatatgaaaaatatggaaacgtgtccgtctttgaaaccactggtggtttggtggtgttctggcaaggtatcaagcaaaaatctctggtggaactcgaacgtttggccaaccgctccagtctgaatcttactcataatagaaccaaaagaagtacagatggcaacaatgcaactcatttatccaacatggagtcggtgcacaatctggtctacgcccagctgcagttcacctatgacacgttgcgcggttacatcaaccgggcgctggcgcaaatcgcagaagcctggtgtgtggatcaacggcgcaccctagaggtcttcaaggaacttagcaagatcaacccgtcagctattctctcggccatctacaacaaaccgattgccgcgcgtttcatgggtgatgtcctgggtctggccagctgcgtgaccattaaccaaaccagcgtcaaggtgctgcgtgatatgaatgtgaaggaatcgccaggacgctgctactcacgaccagtggtcatctttaatttcgccaacagctcgtacgtgcagtacggtcaactgggcgaggataacgaaatcctgttgggcaaccaccgcactgaggaatgtcagcttcccagcctcaagatcttcatcgccggcaactcggcctacgagtacgtggactacctcttcaaacgcatgattgacctcagcagcatctccaccgtcgacagcatgatcgccctagacatcgacccgctggaaaacaccgacttcagggtactggaactttactcgcagaaagaattgcgttccatcaacgtttttgatctcgaggagatcatgcgcgagttcaattcgtataagcagcgggtaaagtacgtggaggacaaggtagtcgacccgctgccgccctacctcaagggtctggacgacctcatgagcggcctgggcgccgcgggaaaggccgttggcgtagccattggggccgtgggtggcgcggtggcctccgtggtcgaaggcgttgccaccttcctcaaaaaccccttcggagccttcaccatcatcctcgtggccatagccgtcgtcattatcatttatttgatctatactcgacagcggcgtctctgcatgcagccgctgcagaacctctttccctatctggtgtccgccgacgggaccaccgtgacgtcgggcaacaccaaagacacgtcgttacaggctccgccttcctacgaggaaagtgtttataattctggtcgcaaaggaccgggaccaccgtcgtctgatgcatccacggcggctccgccttacaccaacgagcaggcttaccagatgcttctggccctggtccgtctggacgcagagcagcgagcgcagcagaacggtacagattctttggacggacagactggcacgcaggacaagggacagaagcccaacctgctagaccgactgcgacaccgcaaaaacggctaccgacacttgaaagactccgacgaagaagagaacgtctga